(19) United States
(12) Patent Application Publication
(10) Pub. No.: US 2026/0116840 A1
(43) Pub. Date: Apr. 30, 2026

(54) MEMBRANE REACTOR FOR CO-GENERATING PROPENE AND ANILINE

(71) Applicant: KING FAHD UNIVERSITY OF PETROLEUM AND MINERALS, Dhahran (SA)

(72) Inventors: Nabeel Salim ABO-GHANDER, Dhahran (SA); Omar Nabeel ALDEEB, Khobar (SA)

(73) Assignee: KING FAHD UNIVERSITY OF PETROLEUM AND MINERALS, Dhahran (SA)

(21) Appl. No.: 18/932,091

(22) Filed: Oct. 30, 2024

(51) Int. Cl. C07C 209/36; C07C 5/333

(52) U.S. Cl. CPC C07C 209/365; C07C 5/333

(57) ABSTRACT

A method of co-generating propene and aniline in an enclosure that includes a shell, and a first membrane positioned inside the shell to separate a hydrogenation side facing the shell and a dehydrogenation side facing away from the shell. The method includes introducing a dehydrogenation gas including propane into the dehydrogenation side via a dehydrogenation inlet, converting the propane to propene and hydrogen in the presence of a dehydrogenation catalyst and diffusing the hydrogen across the first membrane from the dehydrogenation side to the hydrogenation side. The method further includes introducing a hydrogenation gas including nitrobenzene into the hydrogenation side via a hydrogenation inlet and converting the nitrobenzene and the hydrogen to aniline and water vapors in the presence of a hydrogenation catalyst resulting in exothermic heat. The exothermic heat is transferred across the first membrane from the hydrogenation side to the dehydrogenation side.

### BACKGROUND

### Technical Field

The present disclosure is generally directed to a membrane reactor, and in particular, directed towards a membrane reactor for co-generating propene and aniline.

### Description of Related Art

The “background” description provided herein is to present the context of the disclosure generally. Work of the presently named inventors, to the extent it is described in this background section, as well as aspects of the description that may not otherwise qualify as prior art at the time of filing, are neither expressly nor impliedly admitted as prior art against the present invention.

Recently, there has been a major increase in demand for unsaturated hydrocarbons. In particular, unsaturated hydrocarbons including light alkenes such as ethylene and propylene are in demand. In general, unsaturated hydrocarbons are the building blocks of a plurality of essential chemicals. In an example, ethylene and propylene are used as monomers to produce their corresponding polymers (polyethylene and polypropylene). Propylene is also the raw material to produce propylene oxide, acrylonitrile, cumene, butyraldehyde, and acrylic acid.

A popular synthetic route to produce unsaturated hydrocarbons is the dehydrogenation of their corresponding alkanes in the presence of a catalyst. Presently, the majority of propylene is produced as a byproduct from the steam cracking of liquid naphtha and by the fluidized catalytic cracking (FCC) in refineries and on-purpose technologies such as propane dehydrogenation. This increase in popularity of propane dehydrogenation is due to the higher increase in the demand of propylene, as compared to that of ethylene. Hence, a method that specifically produces propylene is required. Another reason for the increasing demand for dehydrogenation techniques to produce ethylene is the construction of more ethane crackers that do not produce any propylene. In addition, the dehydrogenation reaction is endothermic, reversible, and limited by equilibrium, in order to drive the equilibrium forward, the reaction is carried out at high temperatures and nearly atmospheric pressure.

To increase the efficiency of the dehydrogenation reaction used to produce propylene and other unsaturated hydrocarbons, membrane reactors were introduced. The membranes used are permeable to hydrogen, thus allowing it to escape while retaining other products and the reactants. Membrane reactors have been used to produce hydrogen from a variety of reactions. Y. Park and coworkers investigated the use of membrane reactors to produce pure hydrogen gas from ammonia using steam as a sweep gas. H. Lee and coworkers carried out a comparison between hydrogen production from steam methane reforming in a packed-bed reactor, a membrane reactor, and a sorption enhanced membrane reactor. The results showed that the latter increased the production of hydrogen in an environmentally friendly way by adsorbing the carbon dioxide produced by the reaction. Y. Hayakawa and coworkers produced hydrogen from ammonia by a plasma membrane reactor. In an attempt to enhance the hydrogen recovery of the membrane reactor, Sharma R. and coworkers used a multi-pass membrane reactor. Furthermore, P. Tandon and M. Jain studied the decomposition of hydrogen iodide in a non-isothermal packed-ed membrane reactor.

The type of membrane used is also another important aspect of a membrane reactor. There are several options available such as, but not limited to, silica-based membranes and palladium-based membranes. K. Ghasemzadeh and coworkers studied the difference in the performance of Pd—Ag and silica-membrane reactors to produce hydrogen from methanol steam reforming. The study evaluated performance of the reactor by observing the hydrogen yield, carbon monoxide selectivity, and methanol conversion. The conclusion was that the silica membrane reactor was the better choice when compared to both the Pd—Ag membrane reactor and the packed-bed reactor. Moreover, a mathematical model for the dehydrogenation of propane in a membrane reactor was developed, where the outer wall of the reactor was heated, the hydrogen produced on the tube side of the reactor was oxidized in the shell side. The oxidation of hydrogen increased the yield of propylene from 65% in a packed bed reactor to 95% in the membrane reactor. Effect of the reactor configuration was further investigated, the reactor was made of several hydrogen-permeable tubes for the dehydrogenation reaction, placed in a bigger tube. The outer tube (shell) was loaded with a catalyst to carry out the oxidation of hydrogen produced. Moreover, the outer wall was not heated, instead, the temperature inside the reactor was determined by the temperature of the feed. However, the integration between a hydrogenation reaction and a dehydrogenation reaction on either side of the membrane is still ongoing. For example, CN108117116A uses zirconium oxide as a catalyst for converting nitrobenzene to aniline. CN117448744A describes hydrogen-related reactions using a palladium membrane. For example, hydrogen can be generated by dehydrogenating propane to prepare propylene. Hydrogen can used to prepare aniline by hydrogenating nitrobenzene. CN1065852C describes a membrane reactor configuration including a Pd/ceramic composite membrane separating a hydrogenation side (CO+H₂) and a dehydrogenation side (CO+H₂O). Hydrogenation and dehydrogenation catalysts can be dispersed on respective sides of the membrane. CN114933535B describes hydrogenating nitrobenzene into aniline in a liquid phase in a kettle of a film reactor at 100-200 degrees centigrade.

Each of the references discussed above indicates that there is room for further development of a method and an apparatus for the integration of various chemical dehydrogenation and hydrogenation reaction combinations. Accordingly, it is one object of the present disclosure to provide a membrane reactor configuration for simultaneously carrying out the dehydrogenation of propane to propylene and the hydrogenation of nitrobenzene to aniline, by overcoming the thermodynamic limitations of the art.

### SUMMARY

In an exemplary embodiment, a method of co-generating propene and aniline in an enclosure is described. The enclosure includes a shell, a hydrogenation inlet, and a hydrogenation outlet. The enclosure further includes a first membrane positioned inside the shell, the first membrane includes a dehydrogenation inlet and a dehydrogenation outlet. The first membrane separates a hydrogenation side facing the shell and a dehydrogenation side facing away from the shell. The method includes introducing a dehydrogenation gas including propane into the dehydrogenation side via the dehydrogenation inlet. The method further includes converting the propane to propene and hydrogen in the presence of a dehydrogenation catalyst on the dehydrogenation side. The method further includes diffusing the hydrogen across the first membrane from the dehydrogenation side to the hydrogenation side. The method further includes introducing a hydrogenation gas including nitrobenzene into the hydrogenation side via the hydrogenation inlet, converting the nitrobenzene and the hydrogen to aniline and water vapors in the presence of a hydrogenation catalyst on the hydrogenation side, resulting in exothermic heat. Further, the method includes transferring the exothermic heat across the first membrane from the hydrogenation side to the dehydrogenation side.

In some embodiments, the dehydrogenation gas further includes helium, and the converting the propane provides a yield of the propene of from 0.69 to 0.78 at a molar fraction of the helium of from 0.3 to 0.9.

In some embodiments, the converting the propane provides the yield of the propene of from 0.73 to 0.78 at the molar fraction of the helium of from 0.7 to 0.9.

In some embodiments, the method further includes pre-heating the hydrogenation gas to a temperature of from 700 Kelvin (K) to 1000K before the introducing the hydrogenation gas, and the converting the nitrobenzene and the hydrogen provides a conversion of the nitrobenzene of from 0.86 to 0.92 at the temperature of from 700K to 1000K.

In some embodiments, the enclosure further includes a second membrane positioned inside the shell, the second membrane surrounds the first membrane, and the second membrane separates the hydrogenation side from a hydration side.

In some embodiments, the method includes diffusing the water vapors across the second membrane from the hydrogenation side to the hydration side. The second membrane is configured to selectively let the water vapors pass through relative to the aniline.

In some embodiments, the method further includes collecting the propene from the dehydrogenation outlet, collecting the aniline from the hydrogenation outlet, and colleting the water vapors from a hydration outlet of the enclosure.

In some embodiments, the method further includes pre-heating the dehydrogenation gas, the hydrogenation gas or both using the water vapors discharged from the hydration outlet, before introducing the dehydrogenation gas, the hydrogenation gas or both.

In some embodiments, the second membrane includes a ceramic membrane.

In some embodiments, the hydrogenation gas does not include water.

In some embodiments, the first membrane includes a palladium-hydrogen membrane.

In some embodiments, the enclosure includes a plurality of the first membranes positioned inside the shell and spaced apart from each other, and the plurality of the first membranes each includes a respective dehydrogenation inlet and a respective dehydrogenation outlet.

In some embodiments, the method further includes introducing the dehydrogenation gas into the plurality of the first membranes in different directions, including a co-current direction relative to a direction of the hydrogenation gas and a counter-current direction relative to the direction of the hydrogenation gas.

In some embodiments, the method further includes the introducing the dehydrogenation gas into the plurality of the first membranes in the different directions provides a yield of the propene of between 0.75 and 0.78 and a conversion of the nitrobenzene of between 0.90 and 0.94.

In some embodiments, the method further includes pre-heating the dehydrogenation gas to a temperature of from 890K to 1000K before the introducing the dehydrogenation gas.

In some embodiments, the method further includes pressurizing the hydrogenation gas to a pressure of from 1.0 bar to 2.0 bar before the introducing the hydrogenation gas and pressurizing the dehydrogenation gas to a pressure of from 1.0 bar to 2.5 bar before the introducing the dehydrogenation gas.

In some embodiments, the shell is a tubular vessel having the hydrogenation inlet on a first end and the hydrogenation outlet on a second end, and the first membrane is a tubular membrane disposed inside the tubular vessel and axially aligned with the tubular vessel.

In some embodiments, the hydrogenation gas further includes water steam.

In some embodiments, the shell includes a heat-insulating material.

In some embodiments, the enclosure includes a plurality of the first membranes spaced apart from each other in the shell. The enclosure further includes a filler structure filling empty space between the plurality of the first membranes and provide mechanical support for the plurality of the first membranes. The filler structure includes a porous metal support over which the dehydrogenation catalyst is distributed.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings.

FIG. 1A is a schematic perspective diagram of an enclosure having a shell and a first membrane illustrating co-current configuration thereof for flows of hydrogenation and dehydrogenation gases, according to certain embodiments.

FIG. 1B is a schematic perspective diagram of the enclosure illustrating a counter-current configuration thereof for the flows of hydrogenation and dehydrogenation gases, according to certain embodiments.

FIG. 1C is a schematic illustration of the enclosure depicting a second membrane surrounding the first membrane and in conjunction with a heat exchanger, according to certain embodiments.

FIG. 1D is a schematic illustration of the enclosure depicting a plurality of first membranes, according to certain embodiments.

FIG. 2 is a schematic flowchart depicting a method of co-generating propene and aniline, according to certain embodiments.

FIG. 3 is a schematic illustration depicting infinitesimal element on both sides of the enclosure, according to certain embodiments.

FIG. 4 is a performance plot for co-current flows, according to certain embodiments.

FIG. 5 is a performance plot for counter-current flows, according to certain embodiments.

FIG. 6 is a graph depicting comparison between conversion of propane for three reactor configurations, according to certain embodiments.

FIG. 7 is a graph depicting comparison between yield of propene for three reactor configurations, according to certain embodiments.

FIG. 8 is a graph depicting comparison between the performance of the co-current and the counter current configurations, for hydrogenation reaction, according to certain embodiments.

FIG. 9 is a graph depicting comparison between hydrogen flux from co-current reactor configuration and counter current reactor configuration, according to certain embodiments.

FIG. 10 is a graph depicting comparison between heat flux from co-current reactor configuration and counter current reactor configuration, according to certain embodiments.

FIG. 11 is a graph depicting temperature profiles for hydrogenation and dehydrogenation sides of co-current reactor configuration and counter current reactor configuration, according to certain embodiments.

FIG. 12 is a graph depicting effect of molar fraction of helium on the conversion of propane, according to certain embodiments.

FIG. 13 is a graph depicting effect of molar fraction of helium on the conversion of nitrobenzene, according to certain embodiments.

FIG. 14 is a graph depicting hydrogen flux for different mole fractions of helium, according to certain embodiments.

FIG. 15 is a graph depicting the effect of number of tubes on the conversion of propane, according to certain embodiments.

FIG. 16 is a graph depicting the effect of the number of tubes on the conversion of nitrobenzene, according to certain embodiments.

FIG. 17 is a graph depicting the effect of the number of tubes on the heat flux, according to certain embodiments.

FIG. 18 is a graph depicting the effect of number of tubes on the hydrogen flux, according to certain embodiments.

FIG. 19 is a graph depicting temperature profiles for different number of tubes, according to certain embodiments.

FIG. 20 is a graph depicting effect of diameter of the tube on the conversion of propane, according to certain embodiments.

FIG. 21 is a graph depicting the effect of diameter of the tube on the conversion of nitrobenzene, according to certain embodiments.

FIG. 22 is a graph depicting the effect of diameter of the tube on the hydrogen flux, according to certain embodiments.

FIG. 23 is a graph depicting the effect of dehydrogenation feed pressure on the conversion of propane, according to certain embodiments.

FIG. 24 is a graph depicting the effect of dehydrogenation feed pressure on the conversion of nitrobenzene, according to certain embodiments.

FIG. 25 is a graph depicting the effect of dehydrogenation feed pressure on the hydrogen flux, according to certain embodiments.

FIG. 26 is a graph depicting the effect of hydrogenation feed pressure on the conversion of propane, according to certain embodiments.

FIG. 27 is a graph depicting the effect of hydrogenation feed pressure on the conversion of nitrobenzene, according to certain embodiments.

FIG. 28 is a graph depicting the effect of hydrogenation feed pressure on the hydrogen flux, according to certain embodiments.

FIG. 29 is a graph depicting the effect of dehydrogenation feed temperature on the conversion of propane, according to certain embodiments.

FIG. 30 is a graph depicting the effect of dehydrogenation feed temperature on the conversion of nitrobenzene, according to certain embodiments.

FIG. 31 is a graph depicting the effect of dehydrogenation feed temperature on the heat flux, according to certain embodiments.

FIG. 32 is a graph depicting the effect of dehydrogenation feed temperature on the hydrogen flux, according to certain embodiments.

FIG. 33 is a graph depicting the effect of hydrogenation feed temperature on the conversion of propane, according to certain embodiments.

FIG. 34 is a graph depicting the effect of hydrogenation feed temperature on the conversion of nitrobenzene, according to certain embodiments.

FIG. 35 is a graph depicting the effect of hydrogenation feed temperature on the heat flux, according to certain embodiments.

FIG. 36 is a graph depicting the effect of steam to hydrocarbon ratio on the conversion of propane, according to certain embodiments.

FIG. 37 is a graph depicting the effect of steam to hydrocarbon ratio on the conversion of nitrobenzene, according to certain embodiments.

FIG. 38 is a graph depicting the effect of steam to hydrocarbon ratio on the heat flux, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of the present disclosure are directed to a method of co-generating propene and aniline in a reactor configuration where dehydrogenation of propane to propene and hydrogenation of nitrobenzene to aniline occur simultaneously. The above-mentioned reactor configuration may overcome thermodynamic limitations of the dehydrogenation reaction by allowing both mass (hydrogen) and heat transfer between a dehydrogenation side and a hydrogenation side in the reactor configuration. The characteristics of the reactor configuration as disclosed in the present disclosure was analyzed by developing a pseudo-homogeneous reactor model which was solved using the orthogonal collocation method. Further, a parametric study was carried out to determine the effect of a plurality of parameters on conversions of propene and nitrobenzene. The results obtained show that the use of the reactor configuration resulted in an increase in the conversion of propane. Furthermore, the proposed reactor configuration is modular in nature; as such, the present reactor configuration may be used in a co-current configuration favoring dehydrogenation, or in a counter-current configuration favoring hydrogenation. The method and the reactor configuration are designed to improve efficiency and economical aspects of the dehydrogenation and the hydrogenation reactions.

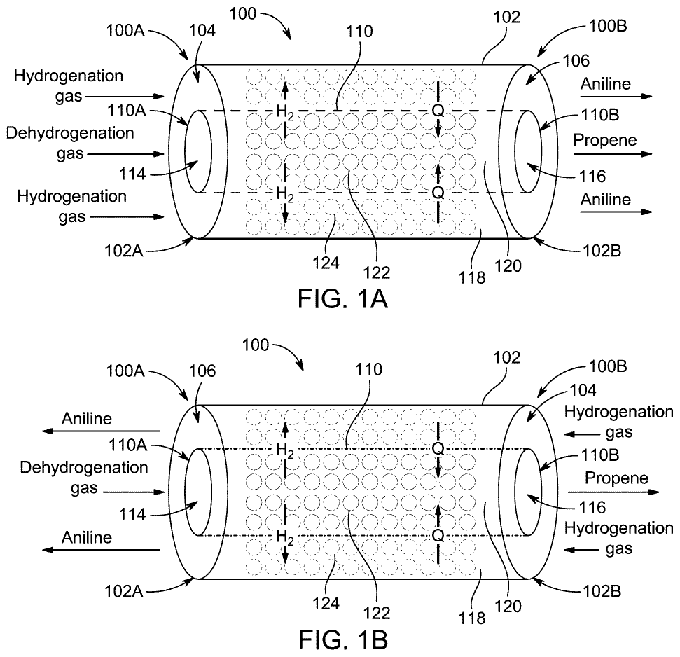

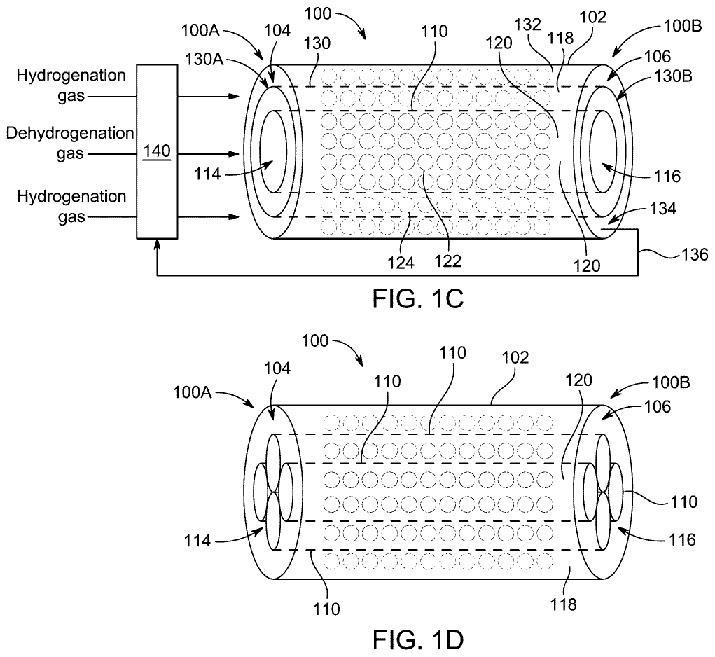

Referring to FIG. 1A, a schematic diagram of an enclosure 100 is illustrated, according to certain embodiments. The enclosure 100 may be interchangeably referred to as “the reactor 100” throughout the description, without any limitations, for the sake of brevity in explanation. In particular, the enclosure 100 can be an adiabatic reactor disposed to provide a reaction space for a dehydrogenation reaction and a hydrogenation reaction. In general, adiabatic reactors are chemical reactors including a thermal insulation disposed on all sides of the reactor. The enclosure 100 includes a shell 102, a hydrogenation inlet 104, and a hydrogenation outlet 106. The shell 102 is thermally insulated in order to impart adiabatic properties to the enclosure 100. In an embodiment, the hydrogenation inlet 104 is configured to be disposed on a first end 100A of the enclosure 100 and the hydrogenation outlet 106 is configured to be disposed on a second end 100B of the enclosure 100. The hydrogenation inlet 104 is configured to supply a hydrogenation gas into the shell 102 and the hydrogenation outlet 106 is configured to discharge a gaseous product generated within the shell 102. In some embodiments, the hydrogenation inlet 104 and the hydrogenation outlet 106 may be defined on the second end 100B and the first end 100A, respectively, of the enclosure 100 depending upon an area of application of the enclosure 100. In some embodiments, the shell 102 is a tubular vessel having the hydrogenation inlet 104 and the hydrogenation outlet 106. In particular, the shell 102 may be manufactured in the form of a tubular vessel, or a hollow cylindrical tube, having a length extending between a first end 102A and a second end 102B thereof. The hydrogenation inlet 104 is defined in the first end 102A of the shell 102 and the hydrogenation outlet 106 is defined in the second end 102B of the shell 102. In some embodiments, the hydrogenation inlet 104 at the first end 102A of the shell 102 may be configured to fluidly tightly couple with an inlet conduit (not shown) to supply the hydrogenation gas into the shell 102 from a hydrogenation gas source. The hydrogenation gas source may be defined as a container configured to store the hydrogenation gas at a desired operating temperature and a desired operating pressure. Similarly, the hydrogenation outlet 106 at the second end 102B of the shell 102 may be configured to fluidly tightly couple with an outlet conduit (not shown) to discharge the gaseous product to an external container. The gaseous product may be stored in the external container at a desired temperature and a desired pressure.

The shell 102 further includers a first membrane 110 positioned inside the shell 102. The first membrane 110 is configured to selectively let hydrogen gas pass through, relative to other gas species such as the aniline, propylene, water vapors, propane and nitrobenzene. The first membrane 110 is also configured to let heat pass through. The first membrane 110 can for example be a palladium-hydrogen membrane. In an embodiment, the first membrane 110 is a tubular membrane disposed inside the tubular vessel, such as the shell 102, and axially aligned with the tubular vessel. In particular, the first membrane 110 may be manufactured in the form of the tubular membrane, or a hollow cylindrical membrane, having a length extending between a first end 110A and a second end 110B thereof. The length of the tubular membrane, or the first membrane 110, may be equal to or less than the length of the tubular vessel, such as the shell 102. Further, the first membrane 110 may be disposed inside the shell 102 in such a way that a central axis of the first membrane 110 may be coaxial to a central axis of the shell 102. The first membrane 110 further includes a dehydrogenation inlet 114 defined at the first end 110A and a dehydrogenation outlet 116 defined at the second end 110B thereof. The dehydrogenation inlet 114 is configured to supply a dehydrogenation gas into the first membrane 110 and the dehydrogenation outlet 116 is configured to discharge a gaseous product generated within the first membrane 110. The first membrane 110 may be further coaxially supported within the shell 102 with the help of mounting brackets and fastening members. As such, the first membrane 110 separates a hydrogenation side 118 facing the shell 102 and a dehydrogenation side 120 facing away from the shell 102. The hydrogenation side 118 may be otherwise defined as a volume defined by an inner diameter of the shell 102 and an outer diameter of the first membrane 110 and the dehydrogenation side 120 may be otherwise defined as a volume defined by an inner diameter of the first membrane 110. In some embodiments, the dehydrogenation inlet 114 at the first end 110A of the first membrane 110 may be configured to fluidly tightly couple with an inlet conduit (not shown) to supply the dehydrogenation gas into the first membrane 110 from a dehydrogenation gas source. The dehydrogenation gas source may be defined as a container configured to store the dehydrogenation gas at a desired operating temperature and a desired operating pressure. Similarly, the dehydrogenation outlet 116 at the second end 102B of the first membrane 110 may be configured to fluidly tightly couple with an outlet conduit (not shown) to discharge a gaseous product to an external container. The gaseous product may be stored in the external container at a desired temperature and a desired pressure.

As shown in FIG. 1A, a co-current configuration of the enclosure 100 is illustrated. In the co-current configuration of the enclosure 100, the hydrogenation inlet 104 and the dehydrogenation inlet 114 are disposed at the first end 100A of the enclosure 100. As such, in the co-current configuration, the dehydrogenation gas including propane is introduced into the enclosure 100 from the first end 100A of the enclosure 100 via the dehydrogenation inlet 114 of the first membrane 110, and the hydrogenation gas including nitrobenzene is also introduced into the enclosure 100 from the first end 100A of the enclosure 100 via the hydrogenation inlet 104. In other words, the hydrogenation gas is introduced into the shell 102 in a co-current direction relative to a direction of the dehydrogenation gas introduced into the first membrane 110. As such, both the hydrogenation gas and the dehydrogenation gas travel in a same direction along a length of the enclosure 100 from the first end 100A to the second end 100B thereof.

As shown in FIG. 1B, a counter-current configuration of the enclosure 100 is illustrated. In the counter-current configuration of the enclosure 100, the dehydrogenation inlet 114 of the first membrane 110 is disposed at the first end 100A of the enclosure 100 and the hydrogenation inlet 104 of the shell 102 is disposed at the second end 100B of the enclosure 100. As such, in the counter-current configuration of the enclosure 100, the dehydrogenation process such as introducing the dehydrogenation gas including the propane into the enclosure 100 from the first end 100A of the enclosure 100 via the dehydrogenation inlet 114 of the first membrane 110 remains similar to that of the co-current configuration, however, the hydrogenation gas including the nitrobenzene is introduced from the second end 100B of the enclosure 100 via the hydrogenation inlet 104 of the shell 102. In other words, the hydrogenation gas may be introduced into the shell 102 in a counter-current direction relative to the direction of the dehydrogenation gas introduced into the first membrane 110. As such, the hydrogenation gas travels in a direction opposite to the direction of the dehydrogenation gas.

In an embodiment of the present disclosure, during a process of co-generating propene and aniline, the dehydrogenation gas including the propane may be introduced into the dehydrogenation side 120 via the dehydrogenation inlet 114 of the first membrane 110. In an embodiment, the dehydrogenation gas may also include helium. Further, the dehydrogenation gas may be pre-heated to a temperature of from 890K to 1000K, preferably 910K to 970K, preferably 930K to 950K, before introducing the dehydrogenation gas into the first membrane 110 of the enclosure 100. The propane may be converted into propene and hydrogen in the presence of a dehydrogenation catalyst 122 on the dehydrogenation side 120. In one embodiment, converting the propane provides a yield of the propene of from 0.69 to 0.78 at a molar fraction of the helium of from 0.3 to 0.9. In another embodiment, converting the propane provides the yield of the propene of from 0.73 to 0.78 at the molar fraction of the helium of from 0.7 to 0.9. The hydrogen generated in the dehydrogenation side 120 may be diffused across the first membrane 110 from the dehydrogenation side 120 to the hydrogenation side 118.

Further, the hydrogenation gas including the nitrobenzene may be introduced into the hydrogenation side 118 via the hydrogenation inlet 104 of the shell 102. In an embodiment, the hydrogenation gas also includes water steam. In an embodiment, the hydrogenation gas may be pre-heated to a temperature of from 700K to 1000K, preferably 750K to 950K, preferably 800K to 900K, preferably 825K to 875K, before introducing the hydrogenation gas into the shell 102 via the hydrogenation inlet 104. The nitrobenzene and the hydrogen may be converted to aniline and water vapors in the presence of a hydrogenation catalyst 124 on the hydrogenation side 118, resulting in exothermic heat. In an embodiment, converting the nitrobenzene and the hydrogen provides a conversion of the nitrobenzene of from 0.86 to 0.92 at the temperature of from 700K to 1000K. The exothermic heat may be further transferred across the first membrane 110 from the hydrogenation side 118 to the dehydrogenation side 120. In some embodiments, the hydrogenation and dehydrogenation gases may be introduced at a pre-determined feed temperature and a pre-determined feed pressure in order to maximize a yield of the enclosure 100. In particular, the hydrogenation gas may be pressurized to a pressure of from 1.0 bar to 2.0 bar, preferably 1.2 bar to 1.8 bar, preferably 1.4 bar to 1.6 bar, before introducing the hydrogenation gas into the shell 102 via the hydrogenation inlet 104, and the dehydrogenation gas may be pressurized to a pressure of from 1.0 bar to 2.5 bar, preferably 1.3 bar to 2.2 bar, preferably 1.6 bar to 1.9 bar, before introducing the dehydrogenation gas into the first membrane 110 via the dehydrogenation inlet 114.

Referring to FIG. 1C, a schematic perspective diagram of the enclosure 100 depicting a second membrane 130 is illustrated, according to certain embodiments. In particular, FIG. 1C shows a configuration of the enclosure 100 in which the second membrane 130 surrounds the first membrane 110. According to the present disclosure, the second membrane 130 includes a ceramic membrane. In some embodiments, the second membrane 130 may be manufactured in the form of a tubular membrane, or a hollow cylindrical membrane, having a length extending between a first end 130A and a second end 130B thereof. The length of the tubular membrane, or the second membrane 130, may be equal to the length of the first membrane 110. Further, the second membrane 130 may be positioned inside the shell 102 in such a way that a central axis of the second membrane 130 may be coaxial to the central axis of the first membrane 110. Further, the second membrane 130 surrounds the first membrane 110 in such a way to separate the hydrogenation side 118 from a hydration side 132. The hydration side 132 may be otherwise defined as a volume defined by the inner diameter of the shell 102 and an outer diameter of the second membrane 130 and the hydrogenation side 118, in this particular configuration of the enclosure 100, may be otherwise defined as a volume defined by an inner diameter of the second membrane 130 and the outer diameter of the first membrane 110. The second membrane 130 is responsible for the hydrogenation reaction, converting nitrobenzene into aniline. In addition, the hydrogenation reaction of nitrobenzene to aniline is exothermic and thermodynamically unlimited. The exothermic nature of the hydrogenation reaction produces heat. In an embodiment, the hydrogenation gas introduced into the enclosure 100 having the second membrane 130 does not include water.

The second membrane 130 is further configured to selectively let the water vapors pass through relative to other gas species such as aniline and nitrobenzene. As such, the water vapors may be diffused across the second membrane 130 from the hydrogenation side 118 to the hydration side 132. During operation, the propene generated in the dehydrogenation side 120 may be collected through the dehydrogenation outlet 116 of the first membrane 110, the aniline generated in the hydrogenation side 118 may be collected through the hydrogenation outlet 106 of the shell 102, and the water vapors diffused across the second membrane 130 may be collected through a hydration outlet 134 defined at the second end 100B of the enclosure 100. The hydration outlet 134 may be further fluid tightly coupled with a water vapor conduit 136 to fluidly communicate the water vapor with a heat exchanger 140. In particular, the heat exchanger 140 is configured to thermally couple with the enclosure 100 via the hydration outlet 134. The heat exchanger 140 is further configured to communicate with the enclosure 100 in such a way to pre-heat the dehydrogenation gas, the hydrogenation gas, or both using the heat of the water vapors discharged from the hydration outlet 134, before the dehydrogenation gas, the hydrogenation gas, or both are introduced into the enclosure 100. In other words, the water vapors from the hydration outlet 134 can be used to pre-heat the dehydrogenation gas, the hydrogenation gas, or both via the heat exchanger 140. The dehydrogenation gas, the hydrogenation gas, or both can then be further heated by one or more external heating sources to desired temperatures as previously discussed. The water vapors exiting the heat exchanger 140 can be collected and stored.

Referring to FIG. 1D, a schematic perspective diagram of a configuration of the enclosure 100 with a plurality of first membranes 110 positioned inside the enclosure 100 is illustrated, according to certain embodiments. The plurality of the first membranes 110 positioned inside the shell 102. While shown to be in contact with each other, the plurality of the first membranes 110 can also be spaced apart from each other. In one embodiment, each of the plurality of first membranes 110 may be coaxially spaced at equal distance from the adjacent first membrane 110. In another embodiment, each of the plurality of first membranes 110 may be coaxially spaced at varying distances from the adjacent first membrane 110 based on the application of the enclosure 100. In some embodiments, each of the plurality of first membranes 110 includes the dehydrogenation inlet 114 and the dehydrogenation outlet 116. As such, the dehydrogenation gas is introduced into the plurality of first membranes 110 in different directions. In particular, the dehydrogenation gas may be introduced into the plurality of first membranes 110 in the co-current direction relative to the direction of the hydrogenation gas, and the counter-current direction relative to the direction of the hydrogenation gas. In an example, the dehydrogenation gas may be introduced from the first end 100A of the enclosure 100 into one first membrane of the plurality of first membranes 110 and the dehydrogenation gas may be introduced into another first membrane of the plurality of first membranes 110 from the second end 100B of the enclosure 100. In some embodiments, introducing the dehydrogenation gas into the plurality of first membranes 110 in the different directions provides a yield of the propene of between 0.75 and 0.78 and a conversion of the nitrobenzene of between 0.90 and 0.94.

In some embodiments, the enclosure 100 includes a filler structure disposed between the plurality of first membranes 110 to fill empty space between the plurality of first membranes 110 thereby to provide mechanical support for the plurality of the first membranes 110. The filler structure includes a porous metal support over which the hydrogenation catalyst is distributed.

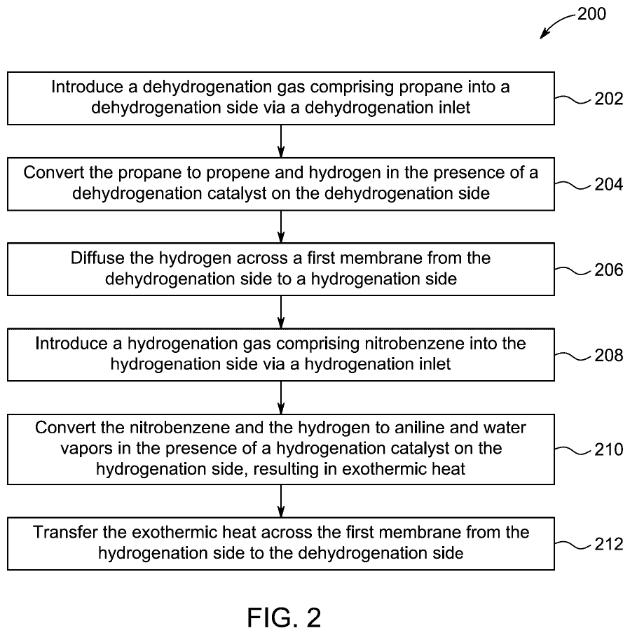

Referring to FIG. 2, a schematic flowchart of a method 200 of co-generating the propene and the aniline in the enclosure 100 is illustrated, according to an embodiment. The order in which the method 200 is described is not intended to be construed as a limitation, and any number of the described method steps may be combined in any order to implement the method 200. Additionally, individual steps may be removed or skipped from method 200 without departing from the spirit and scope of the present disclosure.

At step 202, the method 200 includes introducing the dehydrogenation gas including the propane into the dehydrogenation side 120 via the dehydrogenation inlet 114 of the first membrane 110. The first membrane 110 positioned inside the shell 102 is configured to perform the dehydrogenation reaction, whereas the hydrogenation side 118 defined between the shell 102 and the first membrane 110 is configured to perform the hydrogenation reaction; as such, the enclosure 100 with the concentric arrangement of the shell 102 and the first membrane 110 is configured to generate propene and aniline. In an embodiment, the dehydrogenation gas also includes helium. In some embodiments, the dehydrogenation gas may be pre-heated to a first temperature/a dehydrogenation temperature of from 890K to 1000K, preferably 910K to 980K, preferably 930K to 960K, and pressurized to the pressure of from 1.0 bar to 2.5 bar, preferably 1.3 bar to 2.2 bar, preferably 1.6 bar to 1.9 bar, before introducing the dehydrogenation gas into the first membrane 110 through the dehydrogenation inlet 114. In particular, a heat exchanger, identical to the heat exchanger 140 explained in FIG. 1C, with the configuration of the enclosure 100, having the first membrane 110 and the second membrane 130, may be used to pre-heat the dehydrogenation gas before the dehydrogenation gas is introduced into the first membrane 110. Further, a compressor may be used to pressurize the dehydrogenation gas to 1.0 bar to 2.5 bar, preferably 1.3 bar to 2.2 bar, preferably 1.6 bar to 1.9 bar, before being introduced into the first membrane 110. In some embodiments, a control system may be implemented to control the operation of the heat exchanger and the compressor to control the temperature and the pressure of the dehydrogenation gas based on input parameters such as the desired temperature and the pressure.

At step 204, the method 200 includes converting the propane to the propene and the hydrogen in the presence of the dehydrogenation catalyst 122 on the dehydrogenation side 120. In one embodiment, converting the propane provides the yield of the propene in the range of 0.69 to 0.78 at the molar fraction of the helium of from 0.3 to 0.9. In another embodiment, converting the propane provides the yield of the propene from 0.73 to 0.78 at the molar fraction of the helium from 0.7 to 0.9. In a specific embodiment, when the molar fraction of helium is about 0.9, about 79% of propane is converted to yield about 75-78% of propene, with the balance being hydrogen. Another critical factor affecting the conversion of propane and the percentage yield of propene is the dehydrogenation temperature. Increasing the temperature significantly increases the percentage conversion of propane. In a specific embodiment, at the dehydrogenation temperature of 890 K, the percentage conversion of propane is about 79% to 80% yielding about 77-78% of propene.

At step 206, the method 200 includes diffusing the hydrogen across the first membrane 110 from the dehydrogenation side 120 to the hydrogenation side 118. The first membrane 110 is the permeable tubular membrane, preferably a palladium-hydrogen membrane, which allows the hydrogen generated in the dehydrogenation side, or within the first membrane 110, to pass through the wall of the first membrane 110 into the hydrogenation side 118 of the enclosure 100.

At step 208, the method 200 includes introducing the hydrogenation gas including the nitrobenzene into the hydrogenation side 118 via the hydrogenation inlet 104. In an embodiment, the hydrogenation gas also includes no water steam. In some embodiments, the hydrogenation gas is pre-heated to a second temperature/hydrogenation temperature of from 700K to 1000K, preferably 750K to 950K, preferably 800K to 900K, preferably 825K to 875K, and pressurized to the pressure of from 1.0 bar to 2.0 bar, preferably 1.25 bar to 1.75 bar, preferably 1.5 bar, before introducing the hydrogenation gas into the shell 102 via the hydrogenation inlet 104. As explained in step 202, the heat exchanger may be used to pre-heat the hydrogenation gas before the hydrogenation gas is introduced into the shell 102. Further, a compressor may be used to pressurize the hydrogenation gas to 1.0 bar to 2.5 bar, preferably 1.25 bar to 1.75 bar, preferably 1.5 bar, before the hydrogenation gas is introduced into the shell 102. In some embodiments, the control system may be implemented to control the operation of the heat exchanger and the compressor to control the temperature and the pressure of the hydrogenation gas based on input parameters such as the desired temperature and the pressure of the hydrogenation gas.

In the co-current configuration of the enclosure 100, in which the hydrogenation inlet 104 and the dehydrogenation inlet 114 are defined at the first end 100A of the enclosure, as explained in FIG. 1A, both the dehydrogenation gas and the hydrogenation gas are introduced into the enclosure 100 from the first end 100A of the enclosure 100. In the counter-current configuration of enclosure 100, the hydrogenation inlet 104 is defined at the second end 100B of enclosure 100, and the dehydrogenation inlet 114 is defined at the first end 100A of the enclosure, as explained in FIG. 1B, the dehydrogenation gas is introduced into the enclosure 100 from the first end 100A thereof, and the hydrogenation gas is introduced into the enclosure 100 from the second end 100B thereof.

At step 210, the method 200 includes converting the nitrobenzene and the hydrogen to the aniline and the water vapors (steam) in the presence of the hydrogenation catalyst on the hydrogenation side 118, resulting in exothermic heat. The hydrogenation catalyst is an ammonia synthesis catalyst. When a mole fraction of helium in the dehydrogenation gas is in the range of 0.3-0.9, the percentage conversion of nitrobenzene is in the range of 90-95%. In a specific embodiment, when a mole fraction of helium in the dehydrogenation gas is 0.9, the percentage conversion of nitrobenzene is about 90-91%. Increasing the temperature significantly moderately the percentage conversion of nitrobenzene. In a specific embodiment, at the hydrogenation temperature in the range of 700K to 1000K, the conversion of nitrobenzene is in the range of 0.86-0.92.

At step 212, the method 200 includes transferring the exothermic heat across the first membrane 110 from the hydrogenation side 118 to the dehydrogenation side 120. In some embodiments, as explained in FIG. 1C, the method 200 includes diffusing the water vapors across the second membrane 130 from the hydrogenation side 118 to the hydration side 132. The second membrane 130 disposed inside the shell 102 surrounds the first membrane 110. Further, permeable nature of the second membrane 130 allows the water vapors to pass through a wall of the second membrane 130 into the hydration side 132. In particular, the second membrane 130 is configured to selectively let the water vapors pass through, relative to the aniline and nitrobenzene. In an embodiment, the hydrogenation gas introduced into the enclosure 100 does not include water. The method 200 further includes collecting the propene generated in the dehydrogenation side 120 through the dehydrogenation outlet 116 of the first membrane 110, the aniline generated in the hydrogenation side 118 through the hydrogenation outlet 106 of the shell 102, and the water vapors diffused across the second membrane 130 through the hydration outlet 134. The hydration outlet 134 is fluid tightly coupled with the water vapor conduit 136 to fluidly communicate the water vapor with the heat exchanger 140. The heat exchanger 140 is thermally coupled with the enclosure 100 to pre-heat the dehydrogenation gas, the hydrogenation gas, or both using the heat produced due to the exothermic nature of the hydrogenation rection and water vapors discharged from the hydration outlet 134, before the dehydrogenation gas, the hydrogenation gas, or both are introduced into the enclosure 100.

In some embodiments, the method 200 includes introducing the dehydrogenation gas into the plurality of the first membranes 110 in different directions, including the co-current direction relative to the direction of the hydrogenation gas, as explained in FIG. 1A, and the counter-current direction relative to the direction of the hydrogenation gas, as explained in FIG. 1B. In some embodiments, the dehydrogenation gas is introduced into the plurality of first membranes 110 in different directions including the co-current direction relative to the direction of the hydrogenation gas, and the counter-current direction relative to the direction of the hydrogenation gas, as explained in FIG. 1C.

In some embodiments, the dehydrogenation gas is introduced into the plurality of first membranes 110 in different directions to provide the yield of the propene between 0.75 and 0.78 and the conversion of the nitrobenzene between 0.90 and 0.94.

### EXAMPLES

The following examples demonstrate a method of co-generating propene and aniline in an enclosure. The examples are provided solely for illustration and are not to be construed as limitations of the present disclosure, as many variations thereof are possible without departing from the spirit and scope of the present disclosure.

### Example 1: Enclosure Configuration and Reaction Kinetics

The above-mentioned enclosure may be alternatively referred to as the “reactor”, hereinafter for brevity in explanation. In the reactor configuration, the dehydrogenation of propane is coupled with aniline synthesis in an adiabatic membrane reactor. The reactor configuration, as shown in FIG. 1A and FIG. 1B, can include two concentric tubes, such as the shell 102 and the first membrane 110, containing two different types of catalyst beds. The reactor external wall is assumed to be insulated so that no heat is exchanged between the reactor and the environment. The walls of the first membrane 110 are made up of a highly selective hydrogen membrane which is also heat conductive. On the dehydrogenation side, dehydrogenation of propane to propene takes place and the reaction network is given by Equations 1 to 3:

$$\begin{matrix}{C}_{3} {H}_{8} ↔ {C}_{3} {H}_{6} + {H}_{2} & {ΔH}_{298} = 124. \frac{kJ}{mol}\end{matrix} \qquad ( 1 )$$

$$\begin{matrix}{C}_{3} {H}_{8} ↔ {C}_{2} {H}_{4} + {CH}_{4} & {ΔH}_{298} = 81. \frac{kJ}{mol}\end{matrix} \qquad ( 2 )$$

$$\begin{matrix}{C}_{2} {H}_{4} + {H}_{2} ↔ {C}_{2} {H}_{6} & {ΔH}_{298} = - 137. \frac{kJ}{mol}\end{matrix} \qquad ( 3 )$$

The reaction rate laws of the above reactions on Pt—Sn—K/Al₂O₃ catalyst, expressed in mol/(kg cat×min), are given by Equations 4 to 6:

$$( - {r}_{{C}_{3} {H}_{8} , 1} ) = \frac{{k}_{1} ( {p}_{{C}_{3} {H}_{8}} - \frac{{p}_{{C}_{3} {H}_{6}} {p}_{{H}_{2}}}{{K}_{eq}} )}{1 + \frac{{p}_{{C}_{3} {H}_{8}}}{{K}_{{C}_{3} {H}_{8}}}} \qquad ( 4 )$$

$$( - {r}_{{C}_{3} {H}_{8} , 2} ) = {k}_{2} {p}_{{C}_{3} {H}_{8}} \qquad ( 5 )$$

$$( - {r}_{{C}_{2} {H}_{4} , 3} ) = {k}_{3} {p}_{{C}_{2} {H}_{4}} {p}_{{H}_{2}} \qquad ( 6 )$$

Further, the reaction rate constants are given as functions of temperature by Equations 7 and 8:

$$\begin{matrix}{k}_{i} = {k}_{i 0} exp [ - \frac{{E}_{ai}}{R} ( \frac{1}{T} - \frac{1}{{T}_{0}} ) ] & i ∈ \{ 1 , 2 , 3 \}\end{matrix} \qquad ( 7 )$$

$${K}_{{C}_{3} {H}_{8}} = {K}_{0} exp [ - \frac{Δ H}{R} ( \frac{1}{T} - \frac{1}{{T}_{0}} ) ] \qquad ( 8 )$$

The constants in the above equations are summarized as:

$${k}_{10} = 0.5242 \frac{mol}{kg \cdot bar \cdot min} , {k}_{20} = 0.00465 \frac{mol}{kg \cdot bar \cdot min}$$

$${k}_{30} = 0.000236 \frac{mol}{kg \cdot bar \cdot min} , {K}_{0} = 3.46 ,$$

$${E}_{a 1} = 34.57 \frac{kJ}{mol} , {E}_{a 2} = 137.31 \frac{kJ}{mol} ,$$

$${E}_{a 3} = 154.54 \frac{kJ}{mol} , Δ H = - 85.817 \frac{kJ}{mol} , {T}_{0} = 793.15 K$$

Dehydrogenation of propane is an endothermic and thermodynamically-limited reaction. In a conventional fixed bed reactor, the temperature drops along the reactor due to the established endothermicity. According to Le-Chatelier's principle, the production of propylene may be boosted via operating at low pressure and high temperature. However, in accordance with the present disclosure, palladium-hydrogen membranes are used to permit hydrogen produced on the dehydrogenation side to diffuse to the hydrogenation side. The transportation of hydrogen from the dehydrogenation side to the hydrogenation side promotes the production of propylene.

On the hydrogenation side, a mixture of nitrobenzene and steam is introduced at a suitable feed temperature and pressure ensuring the conversion of nitrobenzene to aniline. Nitrobenzene is converted to aniline according to the following stoichiometric Equation 9:

$$\begin{matrix}{C}_{6} {H}_{5} {NO}_{2} + 3 {H}_{2} \to {C}_{6} {H}_{5} {NH}_{2} + 2 {H}_{2} O & Δ {H}_{298} = - 443 \frac{kJ}{mol}\end{matrix} \qquad ( 9 )$$

The reaction rate expressed in mol/(Kg cat×s) on a palladium catalyst supported on a-alumina carrier is given by Equation 10:

$${r}^{′} = \frac{{k}^{′} {K}_{NB} {K}_{{H}_{2}} {P}_{NB}^{′} \sqrt{{P}_{{H}_{2}}^{′}}}{{( 1 + {K}_{NB} {P}_{NB}^{′} + {K}_{{H}_{2}} \sqrt{{P}_{{H}_{2}}^{′}} )}^{2}} \qquad ( 10 )$$

The reaction rate constant and the adsorption constant of nitrobenzene and hydrogen are estimated by Equations 11, 12 and 13, respectively:

$${k}^{′} = 0.1 \; 86 exp [ - \frac{10.0 * 1 {0}^{- 3}}{{RT}^{′}} ] \qquad ( 11 )$$

$${K}_{NB} = 0.0 \; 151 {kPa}^{- 1} \qquad ( 12 )$$

$${K}_{{H}_{2}} = 0.14 {kPa}^{- 0.5} \qquad ( 13 )$$

The hydrogenation reaction of nitrobenzene to aniline is exothermic and thermodynamically unlimited. In the coupled catalytic membrane reactor of the present disclosure, the heat produced due to the reaction may transfer to the dehydrogenation side to assist in boosting the production of propylene on the dehydrogenation side. This is an example of coupling reactions where both mass and heat are allowed to transfer across the boundary from one side of the reactor to the other, preferably both a majority of the mass and a majority of the heat produced transfer across the boundary from one side of the reactor to the other.

### Example 2: Mathematical Modelling of the Coupled Membrane Reactor

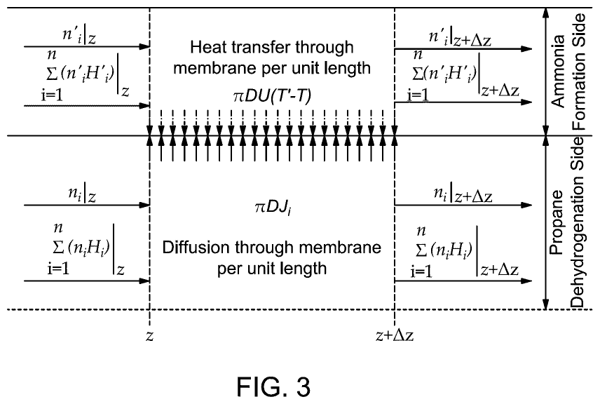

To derive the mathematical model of the coupled membrane reactor, two infinitesimal elements with thicknesses ‘Az’ on both sides of the coupled membrane reactor are considered, as shown in FIG. 3, with mole and energy flow across the two ends of the elements. The two elements interact via hydrogen diffusion and heat transfer between the two sides of the coupled membrane reactor. A plurality of assumptions are considered while deriving the reactor model such as, steady-state operation of the coupled membrane reactor, pseudo-homogeneous reactor model, ideal gas behavior, ideal plug flow, the reactor has a high surface area per unit volume which makes it adiabatically operated, catalyst deactivation is negligible, the hydrogen membrane used is highly selective, hydrogen diffusion through the membrane is accounted for by Savart's law, and the pressure drop on both sides of the reactor is correlated by Ergun's equation.

### Example 3: Mole Energy and Balances

The mole and energy balances on the dehydrogenation side of the reactor are given by Equations 14 and 15, respectively.

$$\frac{{dn}_{i}}{dz} = {\sum }_{j = 1}^{R} {σ}_{ij} {r}_{j} ( 1 - ε ) {A}_{c} {ρ}_{s} - ( π D ) {J}_{i} \qquad ( 14 )$$

$$\frac{dT}{dz} = \frac{π DU ( {T}^{′} - T ) + {{\sum }_{j = 1}}^{R} {[ - Δ H ]}_{j} {r}_{j} ( 1 - ε ) {A}_{c} {ρ}_{s}}{{{\sum }_{i = 1}}^{n} {n}_{i} {C}_{pi}} \qquad ( 15 )$$

The mole and energy balances on the hydrogenation side are given by Equations 16 and 17, respectively.

$$\frac{{dn}_{i}^{′}}{dz} = {( - 1 )}^{a} [ {σ}_{i}^{′} {r}^{′} {ρ}_{s}^{′} ( 1 - {ε}^{′} ) {A}_{c}^{′} + ( π D ) {J}_{i} ] \qquad ( 16 )$$

$$\frac{{dT}^{′}}{dz} = {( - 1 )}^{a} \frac{[ - Δ H ( T ) ] {r}^{′} ( 1 - {ε}^{′} ) {ρ}^{′} {A}_{s}^{′} - π DU ( {T}^{′} - T )}{{\sum }_{i = 1}^{n} {n}_{i}^{′} {C}_{pi}^{′}} \qquad ( 17 )$$

### Example 4: Pressure Drop and Hydrogen Permeation

The pressure drop on both sides of the reactor is accounted for by the Ergun equation 18:

$$\frac{dP}{dz} = - {( - 1 )}^{a} \{ \frac{G}{ρ {D}_{p} {g}_{c}} \frac{1 - ε}{{ε}^{3}} [ \frac{150 ( 1 - ε ) μ}{{D}_{p}} + 1.75 G ] \} \qquad ( 18 )$$

In Equations 16, 17, and 18, the term (−1)^{a} is used to specify the flow configuration of the coupled membrane reactor. “a” possesses a value of 2 for co-current configuration and 1 for countercurrent configuration. A set of boundary conditions are required to solve the above model equations defined for both the dehydrogenation side and hydrogenation side. For the co-current configuration of both sides of the coupled reactor, the boundary conditions on both sides of the reactor are defined at one end only, i.e. z=0. At z=0, the boundary conditions on the dehydrogenation side are given as:

$${n}_{i} = {n}_{i 0}$$

$$T = {T}_{0}$$

$$P = {P}_{0}$$

For the hydrogenation side, it is defined at z=0 as:

$${n}_{i}^{′} = {n}_{i 0}^{′}$$

$${T}^{′} = {T}_{0}^{′}$$

$${P}^{′} = {P}_{0}^{′}$$

For the countercurrent configuration, the boundary conditions are defined at the two ends. For the dehydrogenation side, the boundary conditions are given at z=0 as:

$${n}_{i} = {n}_{i 0}$$

$$T = {T}_{0}$$

$$P = {P}_{0}$$

For the hydrogenation side, however, the boundary conditions are given at z=L as:

$${n}_{i}^{′} = {n}_{i 0}^{′}$$

$${T}^{′} = {T}_{0}^{′}$$

$${P}^{′} = {P}_{0}^{′}$$

Hydrogen permeation rate per unit length in the coupled catalytic membrane reactor is estimated as a function of hydrogen partial pressure between the reactor side and the permeate side according to Sievert's law, as shown in Equation 19:

$${J}_{{H}_{2}}^{′} = \frac{{Q}_{0}}{{δ}_{{H}_{2}}} exp ( - \frac{{E}_{{H}_{2}}}{RT} ) ( \sqrt{{p}_{{H}_{2}}} - \sqrt{{p}_{{H}_{2}}^{′}} ) \qquad ( 19 )$$

$$where :$$

$${Q}_{0} = 7.29 \times {10}^{- 3} \frac{mol}{m \times min \times {atm}^{0.5}} {δ}_{{H}_{2}} = 25.0 \times 1 {0}^{- 6} m$$

$${E}_{{H}_{2}} = 20.5 \times 1 {0}^{3} \frac{J}{mol}$$

Heat exchange rate per unit length between the two sides of the coupled membrane reactor is estimated by considering a series combination of heat transfer resistances including convective heat transfer resistance on the propane side, conductive heat transfer resistance due to the hydrogen membrane, and convective heat transfer resistance on the nitrobenzene side. The mathematical formulation is given by Equation 20:

$${Q}^{′} = \frac{2 π {r}_{1} ( {T}^{′} - T )}{[ \frac{1}{{h}^{′}} + \frac{{r}_{1}}{{k}_{ss}} ln ( \frac{{r}_{2}}{{r}_{1}} ) + \frac{{r}_{1}}{{k}_{Pd}} ln ( \frac{{r}_{3}}{{r}_{2}} ) + \frac{{r}_{1}}{{r}_{2} h} ]} \qquad ( 20 )$$

The convective heat transfer coefficients on both sides of the reactors are given by Equation 21 and 22, respectively:

$$\frac{{hD}_{t}}{{k}_{g}} = 0.8 \; 13 {( \frac{{D}_{p} G}{{μ}_{g}} )}^{0.90} exp ( - \frac{6 {D}_{p}}{{D}_{t}} ) \qquad ( 21 )$$

$$\frac{{h}^{′} {D}_{t}^{′}}{{k}_{g}^{′}} = 3.5 {( \frac{{D}_{p}^{′} {G}^{′}}{{μ}_{g}^{′}} )}^{0.70} exp ( - \frac{4.6 {D}_{p}^{′}}{{D}_{t}^{′}} ) \qquad ( 22 )$$

### Example 5

The method and system of the present disclosure, is normalized, and solved numerically using orthogonal collocation method. The performance of the reactor may be compared under several operation modes such as co-current and counter current. Moreover, the performance of the membrane reactor may be compared to that of the traditional reactor (non-membrane) for the dehydrogenation reaction. In order to successfully assess the differences between the modes of operation, a reference has to be established using the following parameters, number of tubes=1500, tube diameter=0.03 m, shell diameter=2 m, reactor length=5 m, total molar flowrate of steam=15.0, mole fraction of helium=0.9, feed pressure (dehydrogenation-shell)=2.5 bar, feed pressure (hydrogenation-tube)=1.5 bar, feed temperature (dehydrogenation-shell)=890 K, feed temperature (hydrogenation-tube)=1.5 bar, and hydrocarbon to steam ratio=7.5. The parameters including yield of propane, conversion of propane, and conversion of nitrobenzene are used to determine the performance of the reactor.

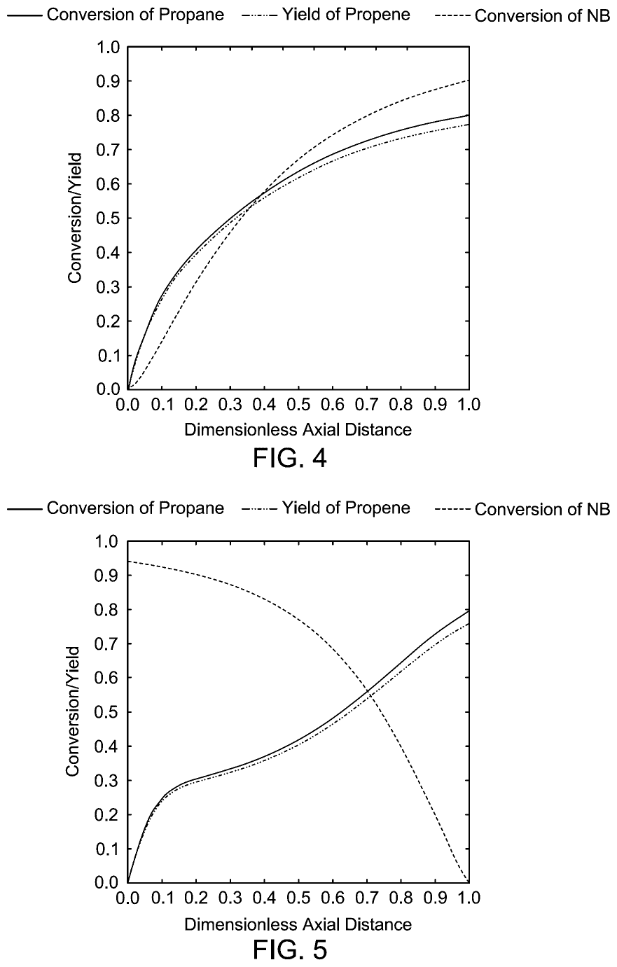

Referring to FIG. 4, a graph depicting the plots of the performance determining parameters is illustrated, according to certain embodiments. The performance determining parameters include conversions of propane and nitrobenzene and the yield of propene, against the dimensionless axial distance for the reference case using a co-current configuration in the membrane reactor. The membrane reactor further operated in a counter current flow such that the dehydrogenation reactions stream was flowing in one direction while as the hydrogenation reaction stream was flowing in the opposite direction. The result obtained using the same parameters as that of the reference case is shown in FIG. 5

### Example 6: Comparison Between the Configuration of the Membrane Reactor and the Conventional Reactor

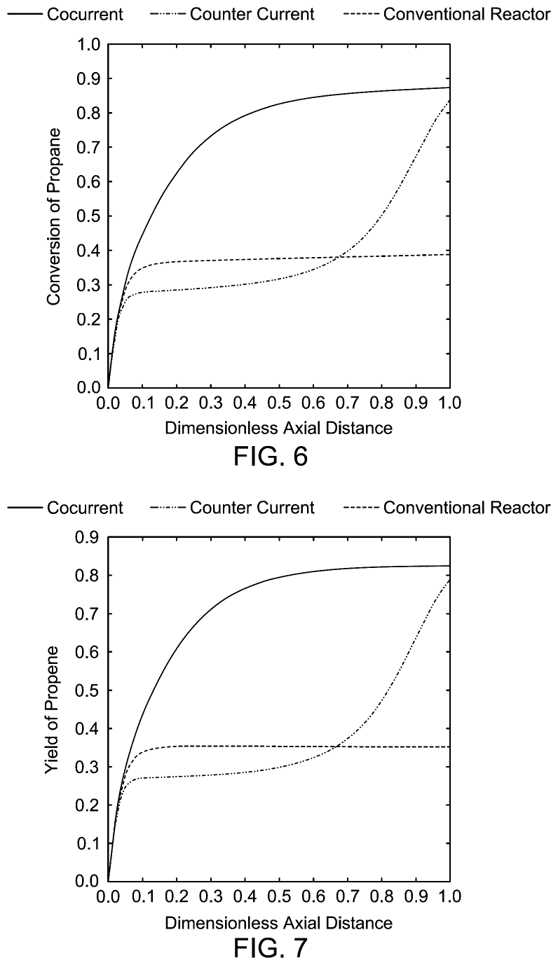

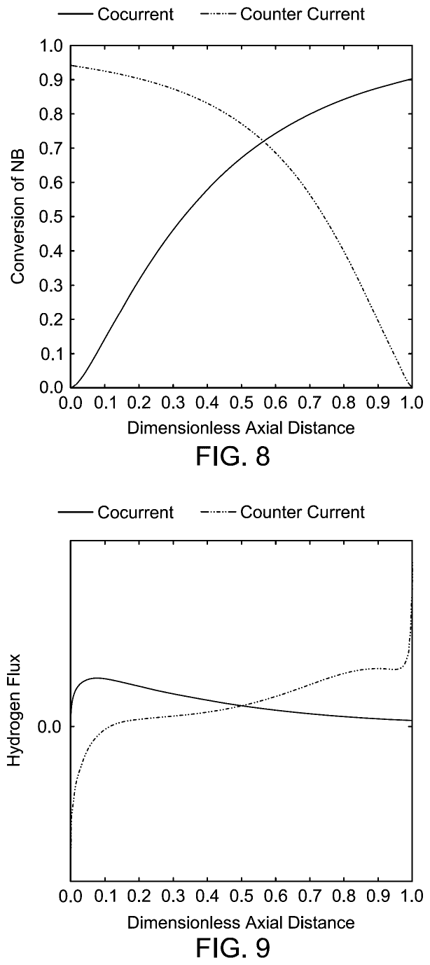

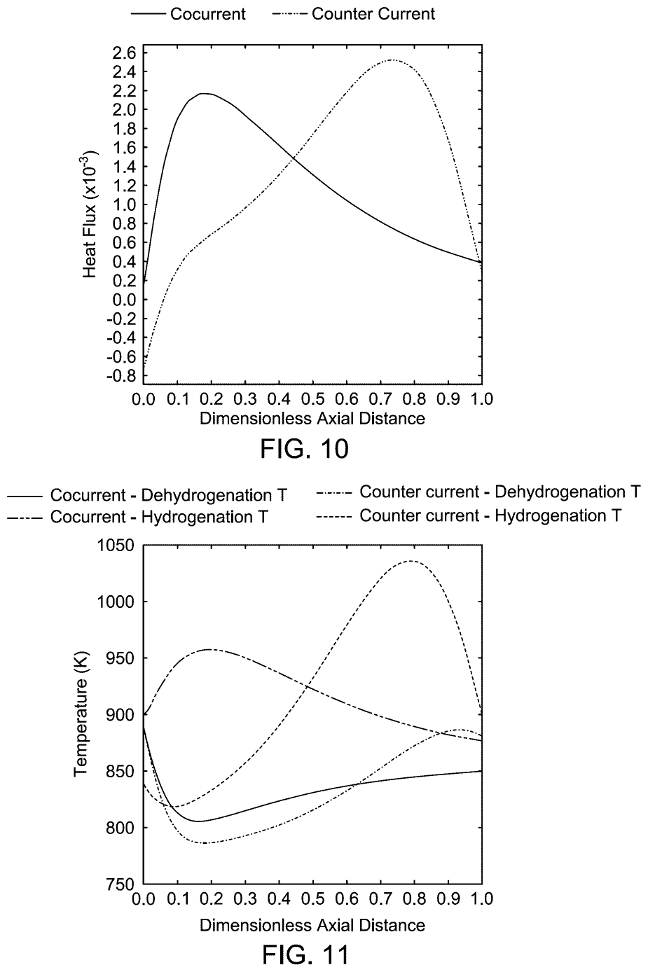

In order to compare the performance of the membrane reactors using co-current and counter current configuration, and that of the conventional reactor (non-membrane), all design and operating variables are kept the same for all three reactor models. The conventional reactor (without a membrane) was modelled by setting the hydrogen flux and the permeability of the membrane to zero. The hydrogenation reaction is turned off and the performance of the dehydrogenation may be observed. FIGS. 6-7 show the comparison of the conversion of propane and yield of propene, respectively. FIGS. 6-7 compare the two configurations with the conventional reactor. Further, FIGS. 8-10 depicts the performance of the hydrogenation side while comparing the co-current and counter current modes. FIG. 8 shows the conversion of nitrobenzene. FIG. 9 and FIG. 10 compare the hydrogen and heat fluxes, respectively. Moreover, the data obtained from the graphs is summarized in Table 1 and the differences in the performances of the reactors were highlighted.

TABLE 1
Comparison between co-current and counter current configurations

| | | | | Percentage |
|---|---|---|---|---|
| | | | | change form co- |
| Performance | Co- | Counter | Conventional | current to |
| parameter | current | Current | Reactor | countercurrent |
| Yield of Propene | 0.7725 | 0.7596 | 0.3541 | −1.67% |
| Conversion of | 0.7996 | 0.7954 | 0.3741 | −0.525% |
| Propane | | | | |
| Conversion of | 0.9022 | 0.941 | — | 4.30% |
| Nitrobenzene | | | | |

As can be seen, transitioning from the conventional reactor to the membrane reactor in either of the configurations (co-current or counter current) more than doubled both the conversion of propane and the yield of propylene. The massive improvement in the performance of the dehydrogenation reactor is due to the heat and hydrogen flux between the shell and the tube in the membrane reactors. The flow of the hydrogen out of the dehydrogenation segment of the reactor through the highly selective hydrogen membrane shifted the propane dehydrogenation reaction to the forward direction which increased its equilibrium conversion. Moreover, the heat flux from the hydrogenation side (exothermic) to the dehydrogenation side (endothermic) makes the reaction more thermodynamically favored thus boosting the conversions of both propane and nitrobenzene. When comparing the performance of both configurations of the membrane reactor, it can be seen that the co-current mode favors the dehydrogenation side at the expense of the hydrogenation side, whereas the counter-current mode favors the hydrogenation side at the expense of the dehydrogenation reaction.

The use of the counter current configuration led to a 4.3% increase in the conversion of nitrobenzene compared to the co-current. However, this increase in the performance of the hydrogenation side causes a decrease in the conversion of propane and the yield of propene of 1.67% and 0.52%, respectively. As shown in FIG. 10, the switch from co-current to counter current led to a higher peak in the heat flux, however, this peak was moved from 0.2 of the reactor length to 0.7 of the reactor length. This shift in the peak of the heat flux favored the conversion of nitrobenzene because the aniline had much higher residence time in the reactor (0.7 until 0) after the peak compared to the propane in the shell (0.7 to 1) since both reactants are moving in opposite directions. This was reflected onto the temperature profile shown in FIG. 11, with the counter current hydrogenation side having a much higher temperature than its counterpart in the co-current configuration. The higher temperature led to a boost in the kinetics of the reaction which in turn led to a higher conversion of nitrobenzene. Also, the dehydrogenation temperature for the counter current flow experienced a lower temperature than that of the co-current flow for over 60% of the reactor which led to lower kinetics and lower equilibrium conversion (endothermic reactor) which led to a decrease in the conversion of propane compared to the co-current configuration. Similarly, the peak for the hydrogen flux has also shifted, as can be seen from FIG. 9, which supports the change in performance between both reactor modes.

### Example 7: Parametric Study

A parametric study for the co-current configuration was carried out to determine the effects of several parameters on the performance of the membrane reactor. To ensure a fair comparison all the parameters were kept the same as that of the reference case and the parameter being tested was varied within their physical limits. The parametric study is important for understanding the reactor.

### Example 8: Molar Fraction of Helium

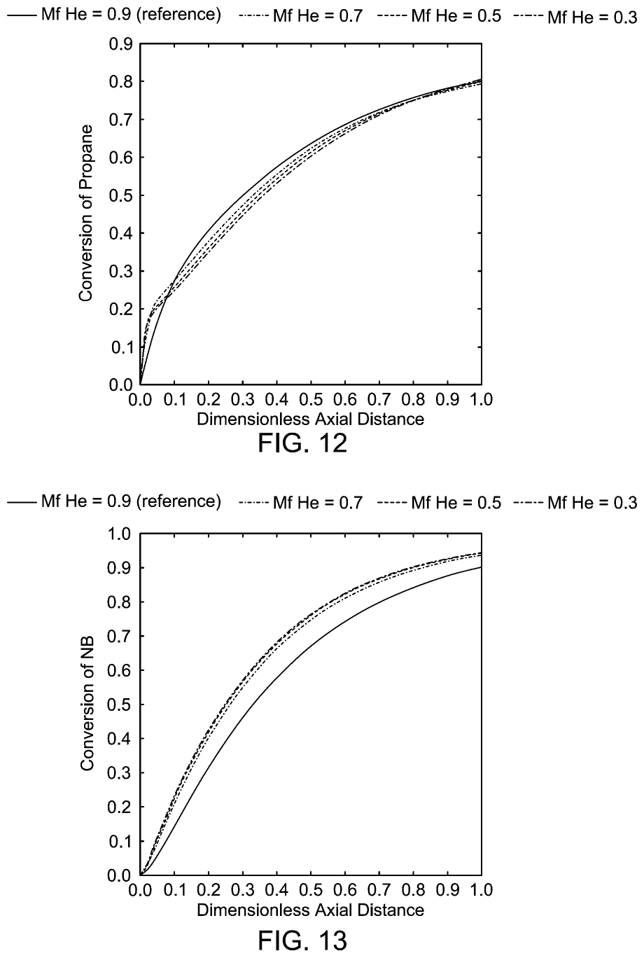

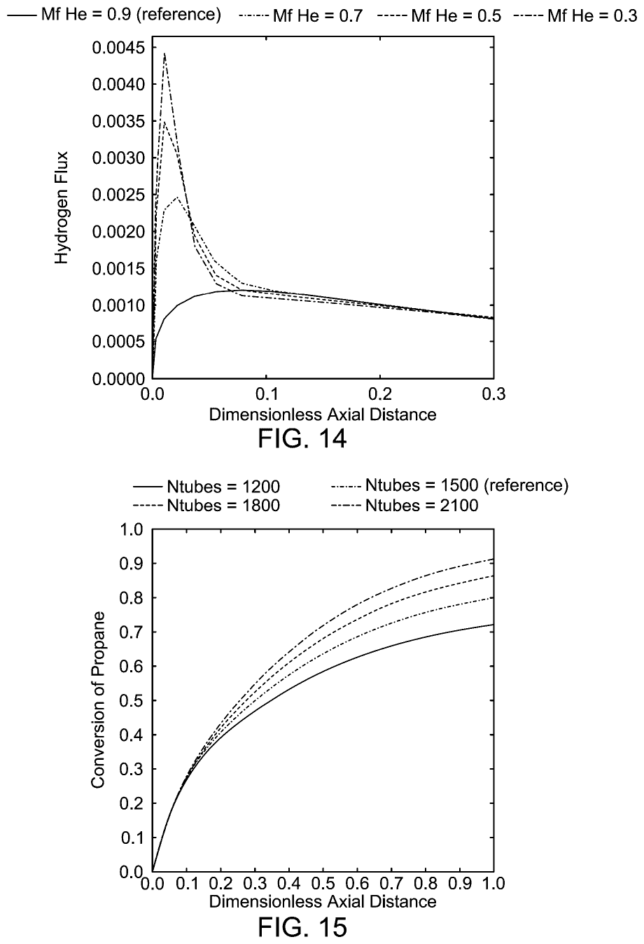

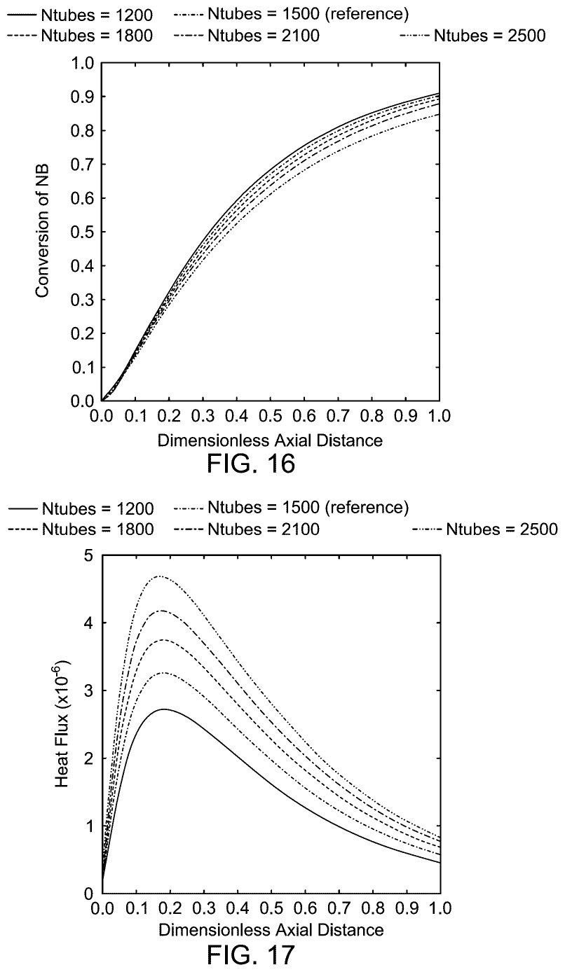

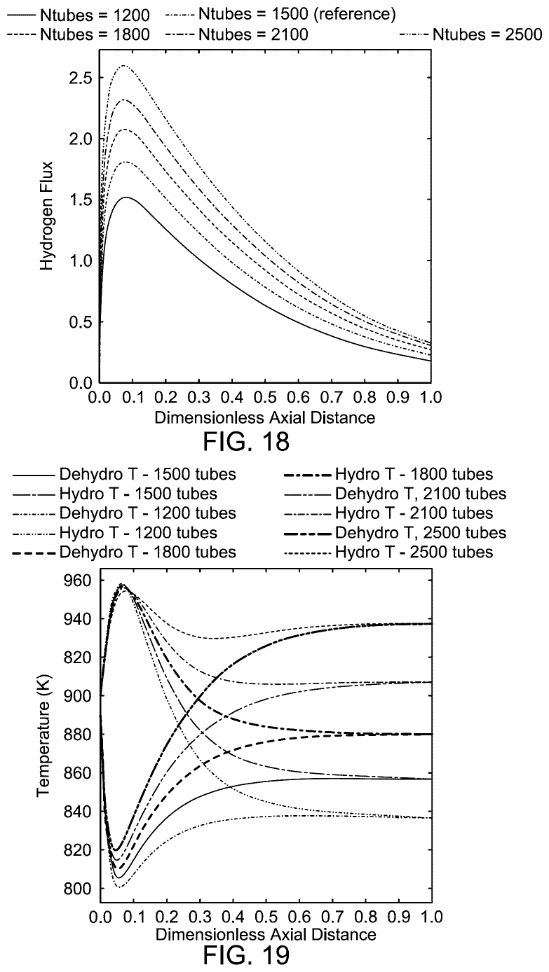

The effect of the molar fraction of helium in the dehydrogenation side (shell) on the performance of the reactor was investigated by varying it from the reference value of 0.9. The molar fractions used were 0.9 (reference), 0.7, 0.5 and 0.3. FIG. 12 shows the effect of the mole fraction of helium on the conversion of propane and FIG. 13 shows its effect on the conversion of nitrobenzene. The increase in the mole fraction of helium from 0.3 to 0.9 diluted the dehydrogenation inlet which decreased the concentration of the reactants which in turn decreased the initial rate of reaction leading to a less steep increase in the conversion of propane. However, the dilution did not significantly change the conversion of propane at the outlet of the reactor. In addition, the dilution of the inlet stream to the dehydrogenation side decreased the partial pressure of hydrogen formed which decreased the hydrogen flux moving to the hydrogenation side, as shown in FIG. 14, which decreased the conversion of nitrobenzene as the mole fraction of helium increased. The conversion of nitrobenzene dropped by 3.7% when the mole fraction of helium increased from 0.7 to 0.9. Table 2 summarizes the results obtained for the different mole fractions of Helium.

TABLE 2
Effect of mole fraction of helium on the
performance of the membrane reactor

| | Mole Fraction of Helium | | | |
|---|---|---|---|---|
| Performance parameter | 0.9 | 0.7 | 0.5 | 0.3 |
| Conversion of Propane | 0.7996 | 0.7930 | 0.7996 | 0.8055 |
| Yield of Propene | 0.7725 | 0.7348 | 0.7122 | 0.6958 |
| Conversion of | 0.9022 | 0.9370 | 0.9426 | 0.9441 |
| Nitrobenzene | | | | |

### Example 8: Number of Tubes

In order to investigate the effect of the number of tubes on the performance of the membrane reactor, the molar flow rate into each tube was kept constant. The numbers of tubes used were 1200, 1500 (reference), 1800, and 2100. FIG. 15 shows the increase in the number of tubes increases the conversion of propane, which may be correlated using FIG. 19, which shows that an increasing number of tubes increases the temperature on the dehydrogenation side of the reactor which according to Le Chatelier's principle pushes the endothermic dehydrogenation reaction given by Equation 1, as mentioned above, to the forward direction and the increase in temperature also increases the rate of the chemical reaction thus increasing the conversion of propane. However, the increase in the number of tubes led to a decrease in the conversion of nitrobenzene in the tubes. This is perhaps because as the temperature on the hydrogenation side drops, the reaction rate of the nitrobenzene drops. Table 3 summarizes the results obtained and the effect of the number of tubes on the performance of the reactor. Increasing the number of tubes by 600 from 1500 to 2100, increased the conversion of propane by 14.1% and slightly decreasing the conversion of nitrobenzene by 2.6%.

TABLE 3
Effect of the number of tubes on the
performance of the membrane reactor

| | |
|---|---|
| Performance | Number of tubes |

| parameter | 1200 | 1500 | 1800 | 2100 |
|---|---|---|---|---|
| Conversion of | 0.7215 | 0.7996 | 0.8637 | 0.9123 |
| Propane | | | | |
| Yield of Propene | 0.6964 | 0.7725 | 0.8346 | 0.8814 |
| Conversion of | 0.9090 | 0.9022 | 0.8925 | 0.8783 |
| Nitrobenzene | | | | |

### Example 9: Tube Diameter

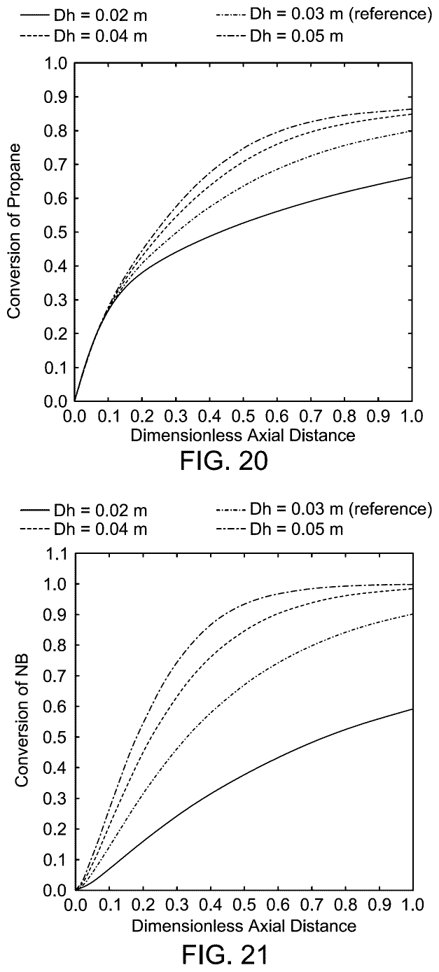

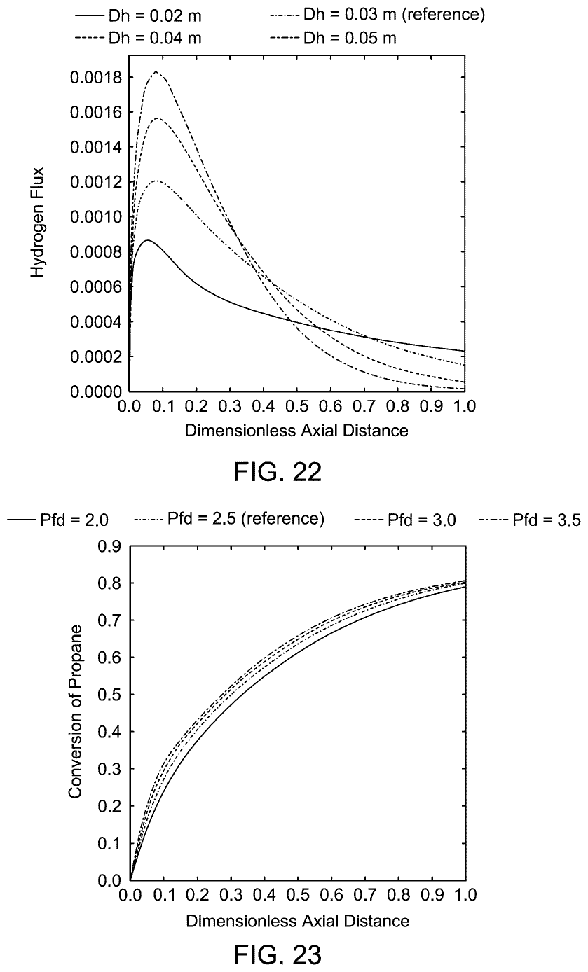

After establishing the effect of the number of tubes, the number of tubes was set back to the reference value of 1500 and the diameter of tubes were changed, to study their effect on the performance of the reactor. The reactor diameters studied were 0.02 m, 0.03 m (reference), 0.04 m and 0.05 m. FIG. 20 shows that as the diameter of the tubes increases, the conversion of propane also increases. Similarly, increasing the diameter of the tube, increases the conversion of nitrobenzene, as shown in FIG. 21. As the tube diameter increases, the cross-sectional area also increases, which leads to an increase in the hydrogen flux through the membrane, as shown in FIG. 21. The increased hydrogen flux decreases the concentration of hydrogen on the dehydrogenation side which shifts the reaction to the forward direction. Consequently, the increase in the hydrogen flux increases the concentration of hydrogen on the hydrogenation side of the reactor which also shifts the hydrogenation reactor to the forward direction. Therefore, the conversions of both propane and nitrobenzene increase. Table 4 shows that doubling the diameter of the tube from 0.02 to 0.04 increases the conversion of propane 28.2% and the conversion of nitrobenzene by 66.2% with nitrobenzene almost reaching complete conversion (0.9845). The results obtained prove that the diameter of the tube has a significant effect on the performance of the reactor boosting the conversion of both reactions.

TABLE 4
Effect of the diameter of the tube on
the performance of the membrane reactor

| | Tube diameter (m) | | | |
|---|---|---|---|---|
| Performance parameter | 0.02 | 0.03 | 0.04 | 0.05 |
| Conversion of Propane | 0.6614 | 0.7996 | 0.8481 | 0.8630 |
| Yield of Propene | 0.6383 | 0.7725 | 0.8187 | 0.8323 |
| Conversion of Nitrobenzene | 0.5923 | 0.9022 | 0.9845 | 0.9983 |

### Example 10: Dehydrogenation Feed Pressure

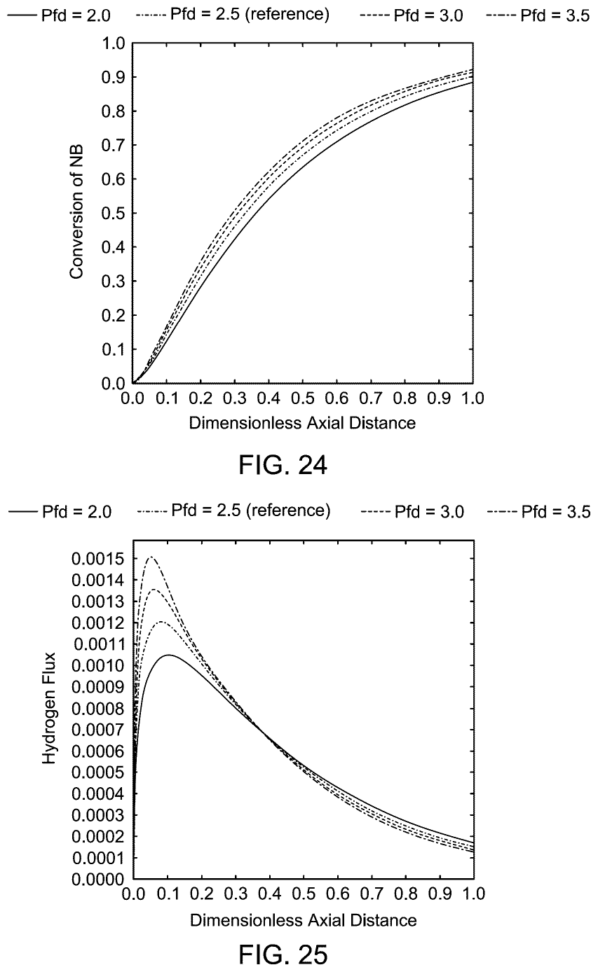

The effect of the hydrogenation and dehydrogenation feed pressures on the performance of the membrane reactor is investigated. The feed pressure on the dehydrogenation side varied between 2.0 bar, 2.5 bar (reference), 3.0 bar and 3.5 bar. According to FIG. 23 and FIG. 24, increasing the feed pressure on the dehydrogenation side increases the conversion of both propane and nitrobenzene, respectively. The increase in the feed pressure on the dehydrogenation side, increases the partial pressure of hydrogen produced on the dehydrogenation side which in turn increases the driving force for hydrogen permeation across the membrane leading to a higher hydrogen flux, as shown in FIG. 25. The higher hydrogen flux between the dehydrogenation and hydrogenation sides shifts both reactions to the forward direction therefore increasing the conversion of their respective reactants. As shown in Table 5, increasing the feed pressure from 2.0 bar to 3.5 bar slightly increases the conversion of propane by 1.95% and the conversion of nitrobenzene increased by 4.18%.

TABLE 5
Effect of the dehydrogenation feed pressure
on the performance of the membrane reactor

| | Feed pressure - dehydrogenation (bar) | | | |
|---|---|---|---|---|
| Performance parameter | 2.0 | 2.5 | 3.0 | 3.5 |
| Conversion of Propane | 0.7909 | 0.7996 | 0.8038 | 0.8064 |
| Yield of Propene | 0.7678 | 0.7725 | 0.7721 | 0.7698 |
| Conversion of Nitrobenzene | 0.8840 | 0.9022 | 0.9134 | 0.9210 |

### Example 11: Hydrogenation Feed Pressure

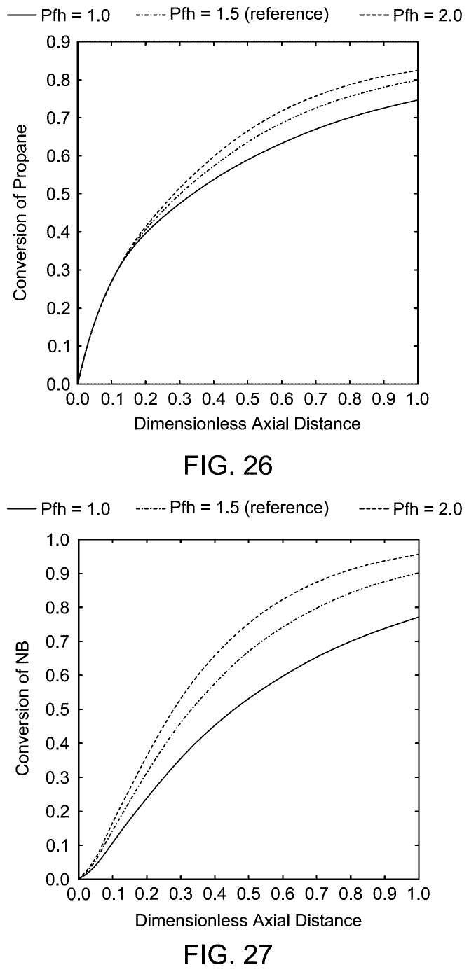

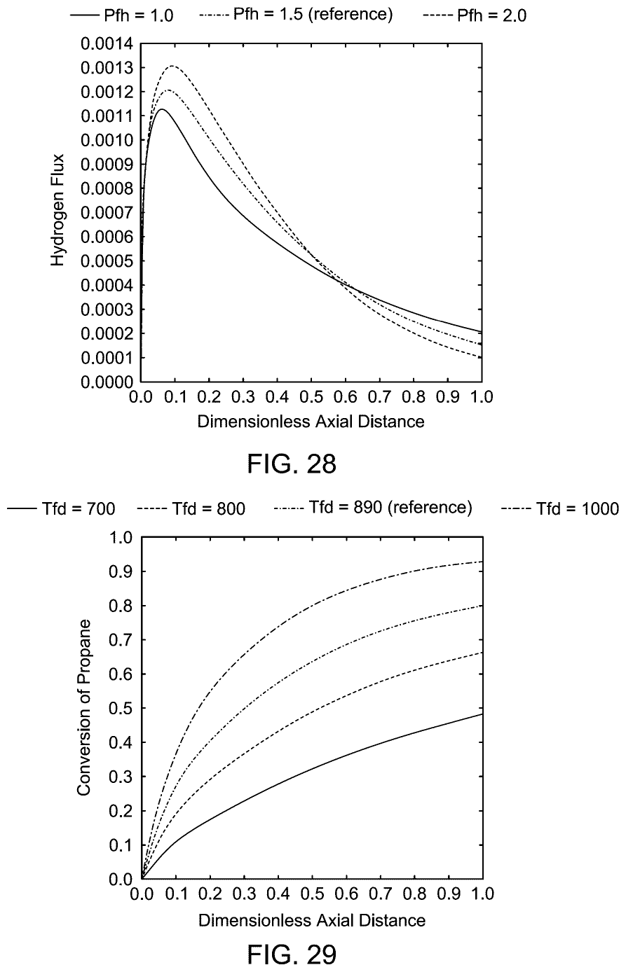

The effect of the hydrogenation feed pressure was investigated. Before selecting the values used for the hydrogenation feed pressure, the pressure on the dehydrogenation side was set to be higher than in the pressure on the hydrogenation side to maintain the flow of hydrogen in the right direction (dehydrogenation to hydrogenation). The reference dehydrogenation feed pressure used was 2.5 bar, so the hydrogenation feed pressures used were 1.0 bar, 1.5 bar (reference) and 2.0 bar. FIGS. 26-27 show the effect of increasing the feed pressure (hydrogenation side) on the conversion of propane and nitrobenzene, respectively. It can be seen that increasing the feed pressure increases both the conversions. Further, FIG. 28 shows that the increase in feed pressure on the hydrogenation side affects the hydrogen flux specially at the reactor inlet. This has led to enhancing the conversion of nitrobenzene to aniline on the hydrogenation side of the coupled membrane reactor. Table 6 summarizes the effects of the change in the feed pressure on the hydrogenation side of the reactor. Doubling the inlet pressure from 1 bar to 2 bar increases the conversion of nitrobenzene by 23.9% while increasing the conversion of propane by 10.2%.

TABLE 6
Effect of the hydrogenation feed pressure
on the performance of the membrane reactor

| | Feed pressure - hydrogenation (bar) | | |
|---|---|---|---|
| Performance parameter | 1.0 | 1.5 | 2.0 |
| Conversion of Propane | 0.7475 | 0.7996 | 0.8239 |
| Yield of Propene | 0.7231 | 0.7725 | 0.7952 |
| Conversion of Nitrobenzene | 0.7711 | 0.9022 | 0.9551 |

### Example 12: Dehydrogenation Feed Temperature

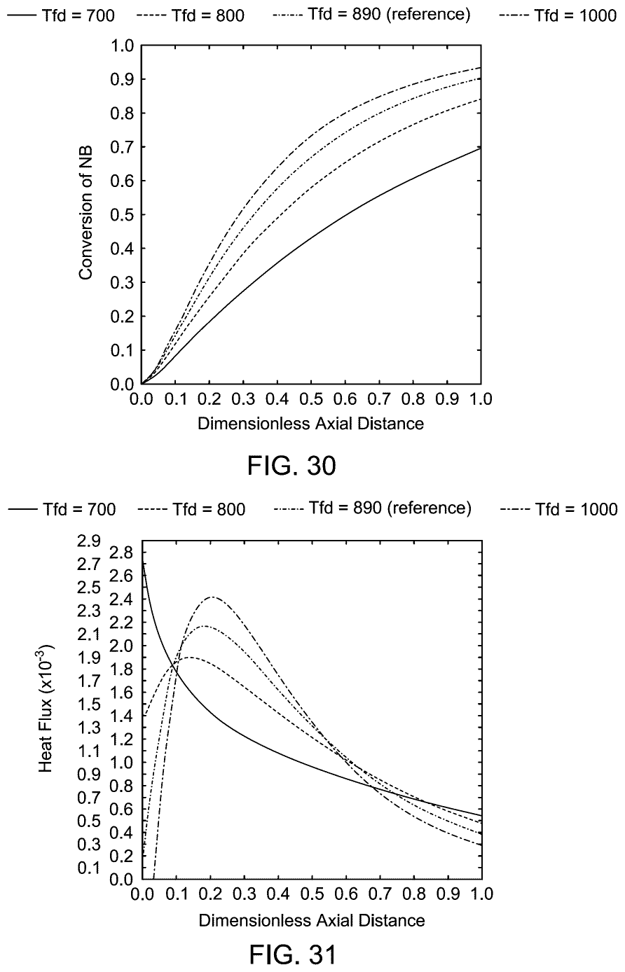

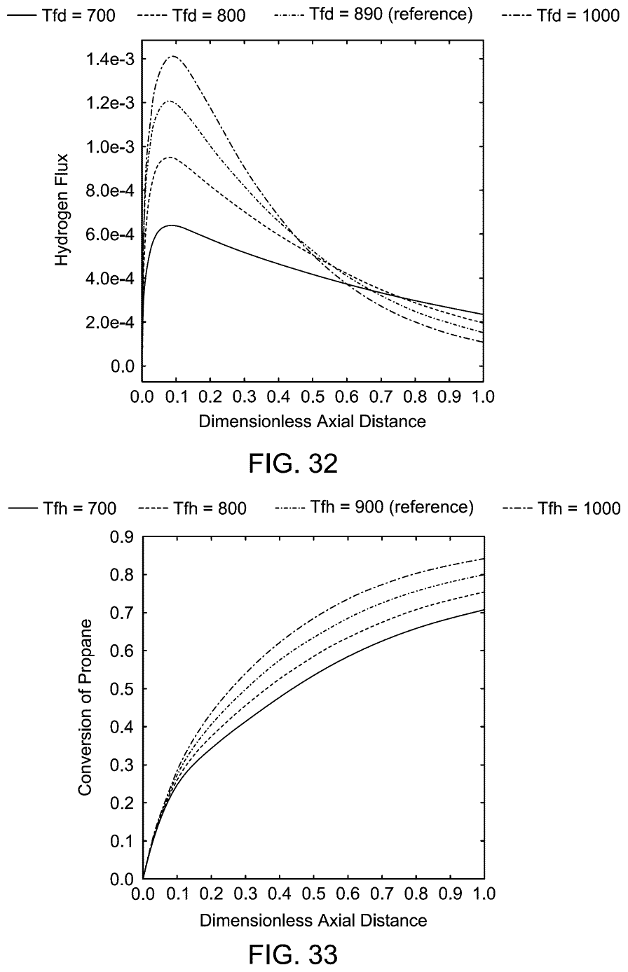

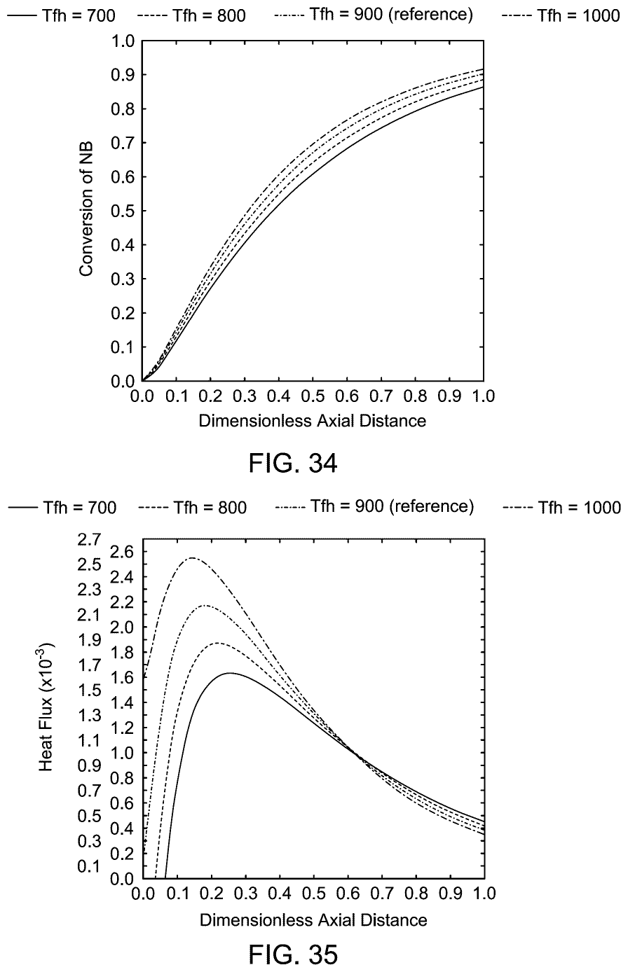

The effect of the feed temperatures was investigated. The dehydrogenation feed temperature was changed to 700 K, 800 K, 890 K (reference) and 1000 K and the effect of the change of temperature on the performance of the reactor was plotted. FIGS. 29-30 show the effect of the feed temperature on the conversion of propane and nitrobenzene, respectively. According to FIGS. 29-30, increasing the feed temperature increases both the conversions of propane and nitrobenzene. The dehydrogenation reaction is endothermic and therefore, increasing the feed temperature shifts the equilibrium of Equation 1 to the forward direction hence increasing the conversion of propane. Further, the increase in the temperature further increases the rate of reaction ensuring the effluent conversion is close to the equilibrium conversion. As the conversion of propane increases, the amount of hydrogen produced also increases, which leads to a higher hydrogen flux through the membrane to the dehydrogenation side, as shown in FIG. 32. The higher hydrogen flux switched the hydrogenation reaction to the forward direction thus increasing the conversion of nitrobenzene. Table 7 summarizes the effect of the dehydrogenation temperature on the performance of the reactor. The increase in the temperature of the dehydrogenation side greatly increased the conversion of propane and slightly increased that of nitrobenzene. Increasing the temperature by 200 K from 800 K to 1000 K increases the conversion of propane by 40.1% and the conversion of nitrobenzene by 10.9%.

TABLE 7
Effect of the dehydrogenation feed temperature
on the performance of the membrane reactor

| | Feed temperature - dehydrogenation (K) | | | |
|---|---|---|---|---|
| Performance parameter | 700 | 800 | 890 | 1000 |
| Conversion of Propane | 0.4833 | 0.6633 | 0.7996 | 0.9297 |
| Yield of Propene | 0.4809 | 0.6534 | 0.7725 | 0.8392 |
| Conversion of Nitrobenzene | 0.6954 | 0.8418 | 0.9022 | 0.9335 |

### Example 13: Hydrogenation Feed Temperature

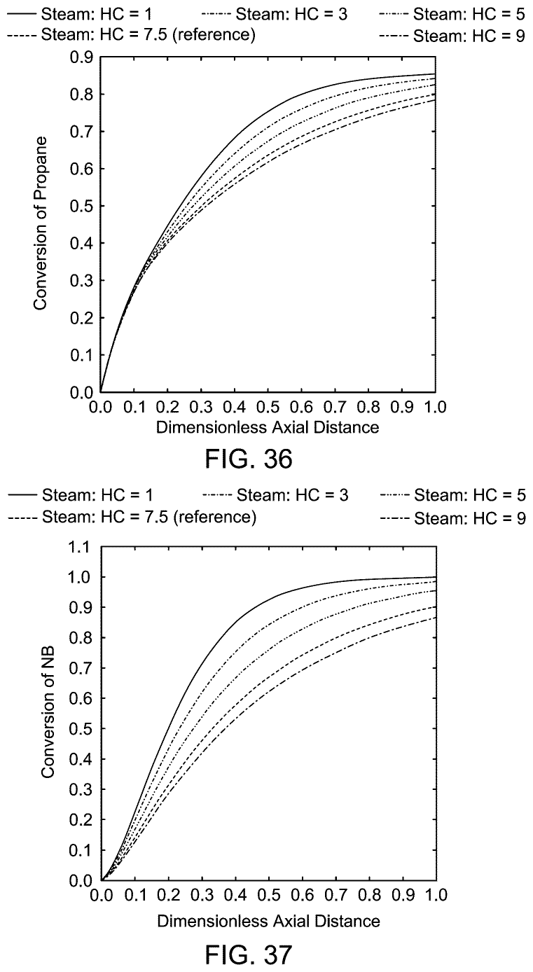

The effect of the feed temperature to the hydrogenation side was investigated. The hydrogenation feed temperature was changed to 700 K, 800 K, 900 K (reference) and 1000 K and the effect of the change of temperature on the performance of the reactor was plotted. FIGS. 36-37 show the effect of the hydrogenation feed temperature on the conversion of propane and nitrobenzene, respectively. According to FIGS. 36-37, increasing the hydrogenation feed temperature increases the conversion of both propane and nitrobenzene. The increase in the conversion of propane is higher than that of nitrobenzene as shown in Table 8 below.

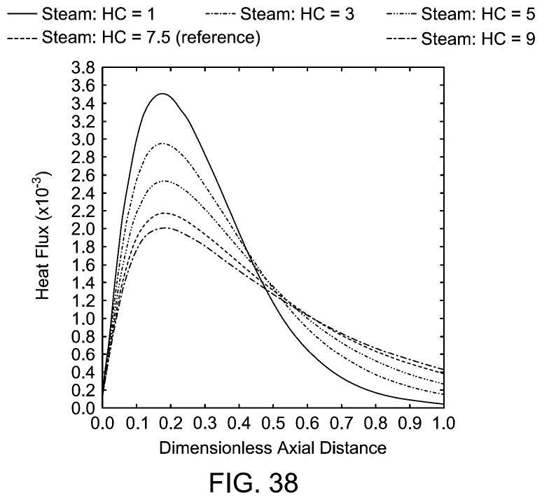

The increase in the conversion of nitrobenzene increases the temperature on the hydrogenation side which increases the driving force for the heat flux leading to a higher heat flux, as shown in FIG. 38. The higher heat flux increases the temperature on the dehydrogenation side shifting its equilibrium to the forward direction leading to an enhancement in the conversion of propane. It is observed that, increasing the hydrogenation feed temperature by 200 K from 800 K to 1000 K, slightly increases the conversion of nitrobenzene by 3.5% while the conversion of propane goes up by 11.7%.

TABLE 8
Effect of the hydrogenation feed temperature
on the performance of the membrane reactor

| | Feed temperature - hydrogenation (K) | | | |
|---|---|---|---|---|
| Performance parameter | 700 | 800 | 900 | 1000 |
| Conversion of Propane | 0.7076 | 0.7543 | 0.7996 | 0.8424 |
| Yield of Propene | 0.6904 | 0.7327 | 0.7725 | 0.8084 |
| Conversion of Nitrobenzene | 0.8637 | 0.8850 | 0.9022 | 0.9161 |

### Example 14: Steam to Hydrocarbon Ratio

On the hydrogenation side, the effect of changing the steam to hydrocarbon ratio in the feed on the performance of the reactor was investigated. The ratio of steam to hydrocarbon was changed by changing the molar flowrate of steam in the hydrogenation feed while keeping the molar flow of nitrobenzene constant to ensure a fair comparison. The steam to hydrocarbon ratios compared were 1, 3, 5, 7.5 (reference) and 9. FIGS. 36-37 show that decreasing the steam to hydrocarbon ratio increases the conversions of both propane and nitrobenzene, respectively. Increasing the steam to hydrocarbon ratio dilutes and decreases the concentration of nitrobenzene leading so a lower rate of reaction leading to decreasing the conversion of nitrobenzene. Furthermore, at a steam to hydrocarbon ratio of 1, the reaction reached its maximum production at roughly 80% of a length of the reactor due to the higher rate of reaction caused by the higher concentration of nitrobenzene in the feed. The decrease in the conversion of nitrobenzene decreases the increase in temperature on the hydrogenation side caused by the exothermic nature of the reaction. The decrease in temperature decreases the heat flux, as shown in FIG. 38, and therefore decreases the temperature increase on the dehydrogenation side compared with lower steam to hydrocarbon ratios. As less heat is transferred from the hydrogenation to the dehydrogenation side and the increase in temperature caused by the flux is lower leading to a lower temperature, the conversion of the endothermic dehydrogenation reaction decreases. Table 9 summarizes the data obtained.

TABLE 9
Effect of the steam to hydrocarbon ratio on
the performance of the membrane reactor

| | Steam to hydrocarbon (NB) ratio | | | | |
|---|---|---|---|---|---|
| Performance parameter | 1 | 3 | 5 | 7.5 | 9 |
| Conversion of Propane | 0.8541 | 0.8421 | 0.8248 | 0.7996 | 0.7842 |
| Yield of Propene | 0.8199 | 0.8109 | 0.7957 | 0.7725 | 0.7580 |
| Conversion of | 0.9978 | 0.9840 | 0.9546 | 0.9022 | 0.8663 |
| Nitrobenzene | | | | | |

The present disclosure provides a method of co-generating propene and aniline. Techniques herein provide a membrane reactor configuration that permits the dehydrogenation of propane to propylene and the dehydrogenation of nitrobenzene to aniline to occur simultaneously. One advantage of the reactor is to boost the thermodynamically limited dehydrogenation of propane by diffusing the hydrogen produced, through a selective membrane, to be utilized to hydrogenate nitrobenzene to aniline. A pseudo-homogeneous reactor model was developed and solved using the orthogonal collocation method. The model generated was used to compare the performance of the membrane reactor with that of the conventional reactor. Further, a parametric study was carried out to determine the effect of several parameters on the conversions of propylene and nitrobenzene. The results obtained showed that the use of the membrane reactor resulted in an increase in the conversion of propane (e.g., about 0.88) when compared to the conventional reactor (e.g., about 0.40) under specific experimental frame as described above. Furthermore, the co-current configuration produced a slightly higher conversion of propane at the expense of a lower conversion of nitrobenzene when compared to the counter current configuration. The parametric analysis showed that increasing the number of tubes, tube diameter, dehydrogenation and hydrogenation feed pressures and temperatures all favored the conversion of propane, whereas the increase in the feed steam to hydrocarbon ratio on the hydrogenation side led to a decrease in the conversion of propane.

Numerous modifications and variations of the present disclosure are possible in light of the above teachings. It is therefore to be understood that within the scope of the appended claims, the invention may be practiced otherwise than as specifically described herein.

1. A method of co-generating propene and aniline in an enclosure that comprises a shell, a hydrogenation inlet and a hydrogenation outlet, the enclosure further comprising a first membrane positioned inside the shell, the first membrane comprising a dehydrogenation inlet and a dehydrogenation outlet, the first membrane separating a hydrogenation side facing the shell and a dehydrogenation side facing away from the shell, the method comprising:
introducing a dehydrogenation gas comprising propane into the dehydrogenation side via the dehydrogenation inlet;
converting the propane to propene and hydrogen in the presence of a dehydrogenation catalyst on the dehydrogenation side;
diffusing the hydrogen across the first membrane from the dehydrogenation side to the hydrogenation side;
introducing a hydrogenation gas comprising nitrobenzene into the hydrogenation side via the hydrogenation inlet;
converting the nitrobenzene and the hydrogen to aniline and water vapors in the presence of a hydrogenation catalyst on the hydrogenation side, resulting in exothermic heat; and
transferring the exothermic heat across the first membrane from the hydrogenation side to the dehydrogenation side.

2. The method of claim 1, wherein:
the dehydrogenation gas further comprises helium, and
the converting the propane provides a yield of the propene of from 0.69 to 0.78 at a molar fraction of the helium of from 0.3 to 0.9.

3. The method of claim 2, wherein:
the converting the propane provides the yield of the propene of from 0.73 to 0.78 at the molar fraction of the helium of from 0.7 to 0.9.

4. The method of claim 1, further comprising:
pre-heating the hydrogenation gas to a temperature of from 700K to 1000K before the introducing the hydrogenation gas,
wherein the converting the nitrobenzene and the hydrogen provides a conversion of the nitrobenzene of from 0.86 to 0.92 at the temperature of from 700K to 1000K.

5. The method of claim 1, wherein:
the enclosure further comprises a second membrane positioned inside the shell,
the second membrane surrounds the first membrane, and
the second membrane separates the hydrogenation side from a hydration side.

6. The method of claim 5, further comprising:
diffusing the water vapors across the second membrane from the hydrogenation side to the hydration side,
wherein the second membrane is configured to selectively let the water vapors pass through relative to the aniline.

7. The method of claim 6, further comprising:
collecting the propene from the dehydrogenation outlet;
collecting the aniline from the hydrogenation outlet; and
colleting the water vapors from a hydration outlet of the enclosure.

8. The method of claim 7, further comprising:
pre-heating the dehydrogenation gas, the hydrogenation gas or both using the water vapors discharged from the hydration outlet, before introducing the dehydrogenation gas, the hydrogenation gas or both.

9. The method of claim 6, wherein:
the second membrane comprises a ceramic membrane.

10. The method of claim 6, wherein:
the hydrogenation gas does not comprise water.

11. The method of claim 1, wherein:
the first membrane comprises a palladium-hydrogen membrane.

12. The method of claim 1, wherein:
the enclosure comprises a plurality of the first membranes positioned inside the shell and spaced apart from each other, and
the plurality of the first membranes each comprises a respective dehydrogenation inlet and a respective dehydrogenation outlet.

13. The method of claim 12, further comprising:
introducing the dehydrogenation gas into the plurality of the first membranes in different directions, including a co-current direction relative to a direction of the hydrogenation gas and a counter-current direction relative to the direction of the hydrogenation gas.

14. The method of claim 13, wherein:
the introducing the dehydrogenation gas into the plurality of the first membranes in the different directions provides a yield of the propene of between 0.75 and 0.78 and a conversion of the nitrobenzene of between 0.90 and 0.94.

15. The method of claim 1, further comprising:
pre-heating the dehydrogenation gas to a temperature of from 890K to 1000K before the introducing the dehydrogenation gas.

16. The method of claim 1, further comprising:
pressurizing the hydrogenation gas to a pressure of from 1.0 bar to 2.0 bar before the introducing the hydrogenation gas; and
pressurizing the dehydrogenation gas to a pressure of from 1.0 bar to 2.5 bar before the introducing the dehydrogenation gas.

17. The method of claim 1, wherein:
the shell is a tubular vessel having the hydrogenation inlet on a first end and the hydrogenation outlet on a second end, and
the first membrane is a tubular membrane disposed inside the tubular vessel and axially aligned with the tubular vessel.

18. The method of claim 1, wherein:
the hydrogenation gas further comprises water steam.

19. The method of claim 1, wherein:
the shell comprises a heat-insulating material.

20. The method of claim 1, wherein:
the enclosure comprises a plurality of the first membranes spaced apart from each other in the shell,
the enclosure further comprises a filler structure filling empty space between the plurality of the first membranes and provide mechanical support for the plurality of the first membranes, and
the filler structure comprises a porous metal support over which the hydrogenation catalyst is distributed.
